(19) United States
(12) Patent Application Publication
(10) Pub. No.: US 2007/0060596 A1
(43) Pub. Date: Mar. 15, 2007

(54) Heterocyclyl compounds

(76) Inventors: Gerard Martin Paul Giblin, Essex (GB); Adrian Hall, Essex (GB); David Nigel Hurst, Essex (GB); Xiao Qing Lewell, Hertfordshire (GB); Olivier Eric Lorthioir, Hertfordshire (GB); Stephen Carl Mickeown, Essex (GB); Tiziana Scoccitti, Essex (GB); Stephen Paul Watson, Hertfordshire (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/576,460

(22) PCT Filed: Oct. 21, 2004
(86) PCT No.: PCT/EP04/11964

§ 371 Date: May 22, 2006

(30) Foreign Application Priority Data
Oct. 24, 2003 (GB) 0324893.7
Oct. 24, 2003 (GB) 0324895.2

(51) Int. Cl. A61K 31/519

(52) U.S. Cl. 514/259.31

(57) ABSTRACT

Compounds of formula (I) or a pharmaceutically acceptable derivative thereof: 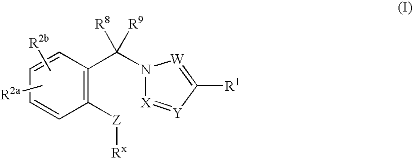
wherein W, X, Y, Z, R¹, R^{2a}, R^{2b}, and R^{x}, R⁸ and R⁹ are as defined in the specification, a process for the preparation of such compounds, pharmaceutical compositions comprising such compounds and the use of such compounds in medicine.

This invention relates to heterocyclic compounds, to processes for their preparation, to pharmaceutical compositions containing them and to their use in medicine, in particular their use in the treatment of conditions mediated by the action of PGE₂ at the EP₁ receptor and conditions mediated by the action of thromboxane on the TP receptor. The invention also relates to compounds having activity at both the EP₁ and TP receptors.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

Certain compounds of the present invention also exhibit antagonism at the TP receptor.

The TP (also known as TxA₂) receptor is a prostanoid receptor subtype stimulated by the endogenous mediator thromboxane. Activation of this receptor results in various physiological actions primarily incurred by its platelet aggregatory and smooth muscle constricting effects, thus opposing those of prostacyclin receptor activation.

TP receptors have been identified in human kidneys (G. P. Brown et al, Prostaglandins and other lipid mediators, 1999, 57, 179-188) in the glomerulus and extraglomerular vascular tissue. Activation of TP receptors constricts glomerular capillaries and suppresses glomerular filtration rates (M. D. Breyer et al, Current Opinion in Nephrology and Hypertension, 2000, 9, 23-29), indicating that TP receptor antagonists could be useful for renal dysfunction in glomerulonephritis, diabetes mellitus and sepsis.

Activation of TP receptors induces bronchoconstriction, increase in microvascular permeability, formation of mucosal oedema and mucus secretion, typical characteristic features of bronchial asthma (T. Obata et al, Clinical Review of Allergy, 1994, 12(1), 79-93). TP antagonists have been investigated as potential asthma treatments resulting in, for example, orally active Seratrodast (AA-2414) (S. Terao et al, Yakugaku Zasshi, 1999, 119(5), 377-390). Ramatroban is another TP receptor antagonist currently undergoing phase III clinical trials as an anti-asthmatic compound.

Antagonists at the TP receptor have been shown to have a gastroprotective effect. In rats it has been shown that SQ 33961 and BM 13505 inhibit gastric lesions induced by taurocholate acid, aspirin or indomethacin (E. H. Ogletree et al, Journal of Pharmacology and Experimental Therapeutics, 192, 263(1), 374-380.

In The American Physiological Society (1994,267, R289-R-294), studies suggest that PGE₂-induced hyperthermia in the rat is mediated predominantly through the EP₁ receptor.

WO 96/06822 (Mar. 7, 1996), WO 96/11902 (Apr. 25, 1996), EP 752421-A1 (Jan. 8, 1997), WO 01/19814 (22 Mar. 2001), WO 03/084917 (16 Oct. 2003), WO 03/101959 (11 Dec. 2003), WO 2004/039753 (13 May 2004) and WO2004/083185 (30 Sep. 2004) disclose compounds as being useful in the treatment of prostaglandin mediated diseases.

P. Lacombe et al (220th National Meeting of The American Chemical Society, Washington D.C., USA, 20-24 Aug. 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists.

It is now suggested that a novel group of pyrazole derivatives surprisingly are selective for the EP₁ receptor over the EP₃ receptor, and are therefore indicated to be useful in treating conditions mediated by the action of PGE₂ at EP₁ receptors. Such conditions include pain, or inflammatory, immunological, bone, neurodegenerative or renal disorders.

It is also suggested that this novel group of pyrazole derivatives are antagonists at the TP receptor and are therefore indicated to be useful in treating conditions mediated by the action of thromboxane at the TP receptor. Such conditions include those disclosed in WO 2004/039807 (Merck Frosst Canada & Co) which is incorporated herein by reference, and include respiratory diseases e.g. asthma, allergic diseases, male erectile dysfunction, thrombosis, renal disorders and gastric lesions.

Accordingly the present invention provides compounds of formula (I): 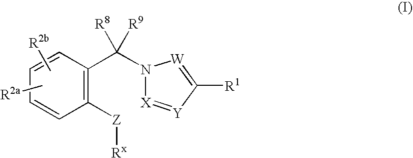
wherein:

W represents N or CR¹⁰ wherein R¹⁰ represents hydrogen, halogen, optionally substituted alkyl, optionally substituted aryl, or optionally substituted heterocyclyl;

X represents N or CR¹¹ wherein R¹¹ represents hydrogen, halogen, optionally substituted alkyl, optionally substituted aryl, or optionally substituted heterocyclyl;

Y represents N or CR¹² wherein R¹² represents hydrogen, halogen, CH₃ or CF₃;

Z represents O, S, SO or SO₂;

R¹ represents CO₂R⁴, CONR⁵R⁶, CH₂CO₂H, optionally substituted SO₂alkyl, SO₂NR⁵R⁶, NR⁵CONR⁵R⁶, 2H-tetrazol-5-yl-methyl or optionally substituted heterocyclyl;

R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;

R^{x} represents optionally substituted alkyl wherein 1 or 2 of the non-terminal carbon atoms are optionally substituted by a group independently selected from NR⁴, O and SOₙ, wherein n is 0, 1 or 2: or R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl;

R⁴ represents hydrogen or an optionally substituted alkyl;

R⁵ represents hydrogen or an optionally substituted alkyl;

R⁶ represents hydrogen or optionally substituted alkyl, optionally substituted heteroaryl, optionally substituted SO₂aryl, optionally substituted SO₂alkyl, optionally substituted So₂heteroaryl, CN, optionally substituted CQ^{a}Q^{b}aryl, optionally substituted CQ^{a}Q^{b}heteroaryl or COR⁷;

R⁷ represents hydrogen, optionally substituted alkyl, optionally substituted heteroaryl or optionally substituted aryl;

R⁸ and R⁹ are independently selected from hydrogen, fluorine or alkyl, or R⁸ and R⁹ together with the carbon to which they are attached form a cycloalkyl ring, optionally containing up to one heteroatom selected from O, S, NH or N-alkyl;

wherein Q^{a} and Q^{b} are each independently selected from hydrogen, CH₃ and fluorine; or derivatives thereof.

Suitably the five membered ring comprising W, X and Y include pyrrole and pyrazole.

Suitably W is CH or N. In one aspect W is N.

Suitably X includes CCH₃, CH and C-thienyl.

Suitably Y includes CH and CF.

Suitably R¹ represents CO₂R⁴. In one aspect R¹ represents CO₂H.

A particular example of Z is O.

When R^{x} represents optionally substituted alkyl this group is preferably C₁₋₈alkyl, for example butyl or isobutyl.

When R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl, suitably R^{x} includes optionally substituted CH₂-heterocyclyl e.g. CH₂-pyridyl, optionally substituted CH₂-bicyclic heterocyclyl or optionally substituted CH₂-aryl e.g optionally substituted CH₂-phenyl. Optional substituents for CH₂-phenyl include one, two or three, preferably one or two substituents selected from Cl, Br, F, CF₃, NO₂, C₁₋₄alkyl and OC₁₋₄alkyl.

Suitably R⁴ includes hydrogen and C₁₋₆alkyl.

Suitably R⁵ includes hydrogen and C₁₋₆alkyl.

Suitably R⁶ includes hydrogen and C₁₋₆alkyl.

Suitably R⁷ includes hydrogen and C₁₋₆alkyl.

Suitably R⁸ includes hydrogen.

Suitably R⁹ includes CH₃ and hydrogen.

Suitably R¹⁰ includes hydrogen.

Suitably R¹¹ includes hydrogen, CH₃ and heterocyclyl, e.g. thienyl.

Suitably R¹² includes hydrogen and halo, e.g. fluorine.

Suitably Q^{a} is hydrogen.

Suitably Q^{b} is hydrogen.

In one aspect, compounds of formula (I) include compounds of formula (Ia):

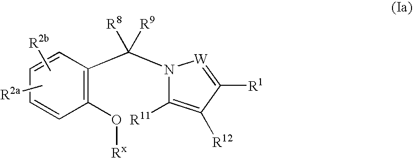

wherein:

W is N or CR¹⁰;

R¹ is CO₂H;

R^{2a} and R^{2b} are independently selected from hydrogen, halo, optionally substituted C₁₋₆alkyl e.g. C₁₋₄alkyl and CF₃, and OC₁₋₆alkyl;

R^{x} is selected from CH₂-pyridyl, C₁₋₆alkyl or CH₂Ph wherein Ph is substituted by R^{3a}, R^{3b} and R^{3c};

R^{3a}, R^{3b} and R^{3c} are independently selected from hydrogen, halo, NO₂, optionally substituted C₁₋₆alkoxy, e.g OCH₃ and optionally substituted C₁₋₆alkyl, e.g CH₃ and CF₃;

R⁸ and R⁹ are independently selected from hydrogen, fluorine or C₁₋₃alkyl, or R⁸ and R⁹ together with the carbon to which they are attached form a C₃₋₆cycloalkyl ring, optionally containing up to one heteroatom selected from O, S, NH or N-C₁₋₆-alkyl;

R¹⁰ is selected from hydrogen, halogen, and optionally substituted C₁₋₈alkyl e.g CH₃ and CF₃;

R¹¹ is selected from hydrogen, halogen, optionally substituted C₁₋₈alkyl e.g. Me and CF₃ and heterocyclyl e.g. thienyl; and

R¹² is selected from hydrogen, halogen e.g. fluorine, and optionally substituted alkyl e.g. CH₃ and CF₃;

or derivatives thereof.

Compounds of formula (I) include the compounds of examples 1 to 61 and derivatives thereof.

The compounds of the invention are selective for EP₁ over EP₃. Preferred compounds are 100 fold selective for EP₁ over EP₃.

Derivatives of the compounds of formula (I) include pharmaceutically acceptable derivatives.

The invention is described using the following definitions unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I).

It will be appreciated that, for pharmaceutical use, the salts referred to above will be pharmaceutically acceptable salts, but other salts may find use, for example in the preparation of compounds of formula (I) and the pharmaceutically acceptable salts thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropyl amine, tromethamine, and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine.

The term “alkyl” as a group or part of a group means a straight, branched or cyclic chain alkyl group or combinations thereof. Unless hereinbefore defined, examples of alkyl include C₁₋₈alkyl, for example methyl, ethyl, n-propyl, iso-propyl, n-butyl, sec-butyl, iso-butyl, t-butyl, pentyl, hexyl, 1,1-dimethylethyl, cyclopentyl or cyclohexyl or combinations thereof such as cyclohexylmethyl and cyclopentylmethyl.

The term “alkoxy” as a group or as part of a group means a straight, branched or cyclic chain alkoxy group. Unless hereinbefore defined “alkoxy” includes C₁₋₈alkoxy, e.g. methoxy, ethoxy, n-propoxy, iso-propoxy, n-butoxy, sec-butoxy,iso-butoxy, t-butoxy, pentoxy, hexyloxy, cyclopentoxy or cyclohexyloxy. In one aspect “alkoxy” is C₁₋₆ alkoxy.

The term “heterocycyl” as a group or as part of a group means an aromatic or non-aromatic five or six membered ring which contains from 1 to 4 heteroatoms selected from nitrogen, oxygen or sulfur and is unsubstituted or substituted by, for example, up to three substituents, preferably one or two substituents. Examples of 5-membered heterocyclyl groups include furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl or tetrazolyl. Examples of 6-membered heterocyclyl groups are pyridyl, pyridazinyl, pyrimidinyl, pyrazinyl, triazinyl or tetrazinyl.

The term “aryl” as a group or part of a group means a 5 or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents, for example up to 3 or up to 2 substituents. Examples of “heteroaryl” include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

When the heteroatom nitrogen replaces a carbon atom in an alkyl group, or when nitrogen is present in a heteroaryl, heterocyclyl or bicyclic heterocyclyl group, the nitrogen atom will, where appropriate be substituted by one or two substituents selected from hydrogen and C₁₋₈alkyl, preferably hydrogen and C₁₋₆alkyl, more preferably hydrogen.

Optional substituents for alkyl groups unless hereinbefore defined include OH, CO₂H, CO₂C₁₋₆alkyl, NHC₁₋₆alkyl, NH₂, (O), OC₁₋₆alkyl, phenyl or halo e.g. Cl, Br or F. An alkyl group may be substituted by one or more optional substituents, for example up to 5, 4, 3, 2 or 1 optional substituents. In one aspect substituted alkyl groups include those substituted by one or more fluorine atoms, up to per-fluorination, e.g. CF₃.

Optional substituents for alkoxy groups unless hereinbefore defined include OH, and halo e.g. Cl, Br or F. An alkoxy group may be substituted by one or more optional substituents, for example up to 5, 4, 3, or 2 optional substituents.

Unless otherwise defined, optional substituents for aryl, heteroaryl or heterocyclyl moieties as a group or part of a group are selected from C₁₋₆alkyl, C₁₋₆alkoxy and halogen.

Compounds of formula (I) can be prepared as set forth in the following schemes and in the examples. The following processes form another aspect of the present invention.

For example, compounds of formula (I) may be prepared by the general route below: 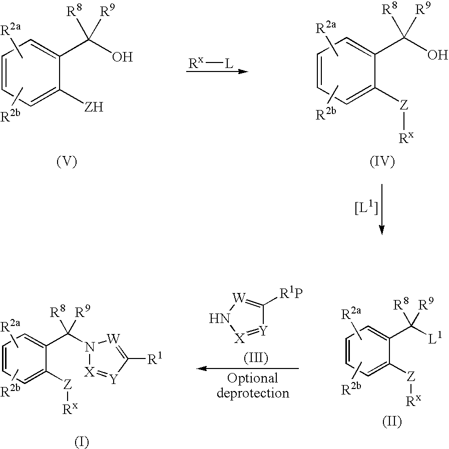
wherein L and L¹ are leaving groups, for example halo e.g. bromo; and W, X, Y, Z, R^{2a}, R^{2b}, R¹, R⁸, R⁹, and R^{x} are as defined for compounds of formula (I), and P is an optional protecting group. The skilled person will recognise when the use of a protecting group is necessary. When R¹ is CO₂H, R¹P is suitably CO₂C₁₋₄alkyl or optionally substituted benzyl.

Suitable reaction conditions for the reaction of an azole of formula (III) with a compound of formula (II) to give a compound of formula (I) include heating in a solvent, e.g. ethanol, in the presence of a base, e.g. potassium tert-butoxide.

Suitable reaction conditions for the preparation a compound of formula (II) include conventional methods for converting the hydroxy group of the compound of formula (IV) to a leaving group, for example when L¹ is Br, the compound of formula (IV) may be reacted with phosporous tribromide in a solvent, e.g. dichloromethane, at reduced temperatures, e.g. less than −10° C.

Suitable reaction conditions for the reaction of a compound of formula (V) with a compound R^{x}-L to give a compound of formula (IV) are known to those skilled in the art and include the use of a solvent e.g. a C₁₋₄alcohol such as methanol or ethanol in the presence of a base, e.g. sodium hydroxide. The skilled person will appreciate that when Z is SO or SO₂, the alkylation step is carried out when Z is S, and the sulfur is then oxidised to the required oxidation state by conventional means at an appropriate stage in the synthesis.

Accordingly the present invention also provides a process for the preparation of a compound of formula (I) or a derivative thereof: 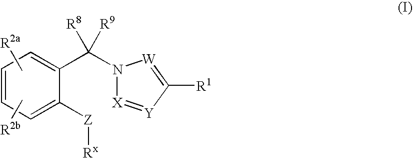
wherein:

W represents N or CR¹⁰ wherein R¹⁰ represents hydrogen, halogen, optionally substituted alkyl, optionally substituted aryl, or optionally substituted heterocyclyl;

X represents N or CR¹¹ wherein R¹¹ represents hydrogen, halogen, optionally substituted alkyl, optionally substituted aryl, or optionally substituted heterocyclyl;

Y represents N or CR¹² wherein R¹² represents hydrogen, halogen, CH₃ or CF₃;

Z represents O, S, SO or SO₂;

R¹ represents CO₂R⁴, CONR⁵R⁶, CH₂CO₂H, optionally substituted SO₂alkyl, SO₂NR⁵R⁶, NR⁵CONR⁵R⁶, 2H-tetrazol-5-yl-methyl or optionally substituted heterocyclyl;

R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;

R^{x} represents optionally substituted alkyl wherein 1 or 2 of the non-terminal carbon atoms are optionally substituted by a group independently selected from NR⁴, O and SOₙ, wherein n is 0, 1 or 2: or R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl;

R⁴ represents hydrogen or an optionally substituted alkyl;

R⁵ represents hydrogen or an optionally substituted alkyl;

R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;

R⁷ represents hydrogen, optionally substituted alkyl, optionally substituted heteroaryl or optionally substituted aryl;

R⁸ and R⁹ are independently selected from hydrogen, fluorine or alkyl, or R⁸ and R⁹ together with the carbon to which they are attached form a cycloalkyl ring, optionally containing up to one heteroatom selected from O, S, NH or N-alkyl;

wherein Q^{a} and Q^{b} are each independently selected from hydrogen, CH₃ and fluorine;

comprising:

reacting a compound of formula (II):

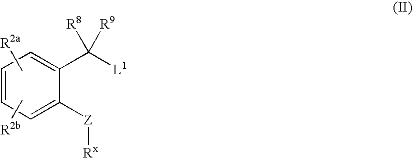

wherein L¹ is a leaving group and Z, R⁸, R⁹, R^{2a}, R^{2b}, and R^{x} are as defined above for a compound of formula (I);

with a compound of formula (III):

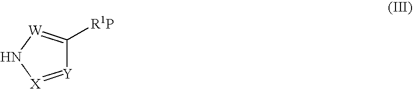

wherein W, X, Y, and R¹ are as defined above for a compound of formula (I) and P is an optional protecting group;

and where required, and in any order;

interconverting one substituent to another substituent; and/or

if necessary removing the optional protecting group; and/or

forming a derivative thereof.

Compounds of formula (I) wherein Z is O, W is N, X is CR¹¹, Y is CR¹², and R¹ is COOH may be prepared by the general route below.

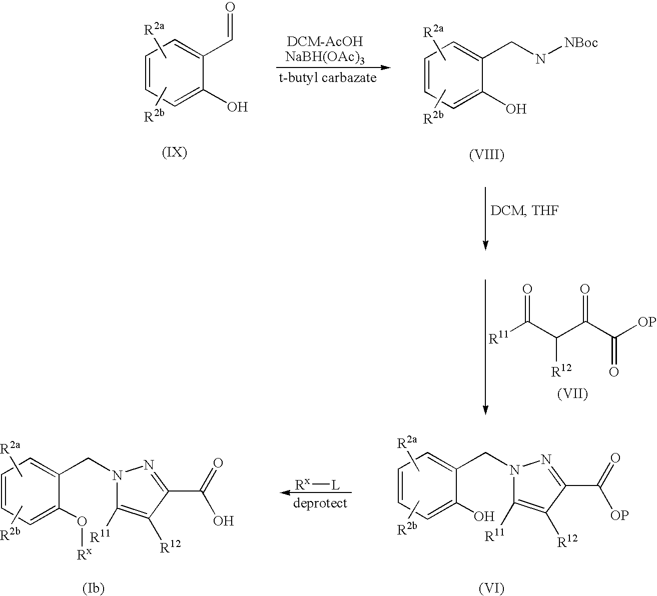

wherein L is a leaving group for example halo, e.g. bromo; P is a protecting group for example C₁₋₄ alkyl e.g. methyl or ethyl; and R^{2a}, R^{2b}, R¹¹, R¹² and R^{x} are as defined for compounds of formula (Ia).

Accordingly the present invention also provides a process for the preparation of a compound of formula (Ib) or a derivative thereof:

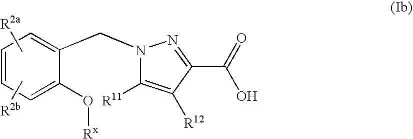

wherein:

R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;

R^{x} represents optionally substituted alkyl wherein 1 or 2 of the non-terminal carbon atoms are optionally substituted by a group independently selected from NR⁴, O and SOₙ, wherein n is 0, 1 or 2: or R^{x} may be optionally substituted CQ^{a}Q^{b}-heterocyclyl, optionally substituted CQ^{a}Q^{b}-bicyclic heterocyclyl or optionally substituted CQ^{a}Q^{b}-aryl;

R⁴ represents hydrogen or an optionally substituted alkyl;

R⁵ represents hydrogen or an optionally substituted alkyl;

R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;

R⁷ represents hydrogen, optionally substituted alkyl, optionally substituted heteroaryl or optionally substituted aryl;

R¹¹ represents hydrogen, halogen, optionally substituted alkyl, optionally substituted aryl, or optionally substituted heterocyclyl; and

R¹² represents hydrogen, halogen, CH₃ or CF₃;

wherein Q^{a} and Q^{b} are each independently selected from hydrogen, CH₃ and fluorine;

comprising:

reacting a compound of formula (VI):

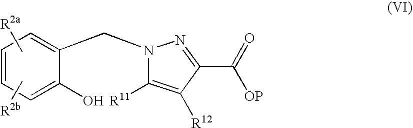

wherein R^{2a}, R^{2b}, R¹¹ and R¹² are as defined above for a compound of formula (Ib) and P is a protecting group;

with R^{x}-L wherein R^{x} is as defined for compounds of formula (I) and L is a leaving group;

and where required, and in any order

interconverting one substituent to another substituent; and/or

removing the protecting group; and/or

forming a derivative thereof.

When one or both of R¹¹ and R¹² is/are halogen, preferably the halogen group is introduced after the ring forming reaction of a compound of formula (VII) and (VIII).

Suitable fluorination conditions are described in e.g. K. Makino et al, J. Fluor. Chem, 1988, 39, 435440. Halogenation conditions are also reviewed in e.g. Comprehensive heterocyclic chemistry. The structure, reactions, synthesis and uses of heterocyclic compounds, A. R. Katritzky and C. W. Rees (Eds), vols 1-8, Pergamon Press, Oxford, 1984; Comprehensive organic chemistry II. A review of the literature 1982-1995, A. R. Katritzky, C. W. Rees, and E. F. V. Scriven (eds), vols 1-11, Pergamon Press, Oxford, 1996, and Heterocyclic Chemistry, 4th Edition, J. A. Joule and K. Mills, Blackwell Science, 2000.

Suitable reaction conditions for the reaction of a compound of formula (VI) with a compound R^{x}-L are known to those skilled in the art and include the use of a solvent e.g. a C₁₋₄alcohol such as methanol or ethanol in the presence of a base, e.g. sodium hydroxide. Suitable conditions for the deprotection of an ester to give the corresponding carboxylic acid are known to those skilled in the art.

Suitable reaction conditions for the reaction of a compound of formula (VII) with a compound of formula (VIII) to give a pyrazole of formula (VI) will be apparent to the skilled person and include treatment with trifluoroacetic acid in a solvent, e.g. dichloromethane, at room temperature to remove the protecting group on the compound of formula (VIII) followed by condensation with (VII) in a solvent such as acetic acid or an alcohol such as methanol.

Suitable reaction conditions for the conversion of a salicylaldehyde of formula (IX) to a compound of formula (VIII) include reacting the salicylaldehyde with tert-butyl carbazate in the presence of acetic acid and sodium triacetoxyborohydride in a solvent such as dichloromethane.

Compounds of formula (I) wherein Z is O, W is CR¹⁰, X is CR¹¹, Y is CR¹², and R¹ is COOH may be prepared by the general route below:

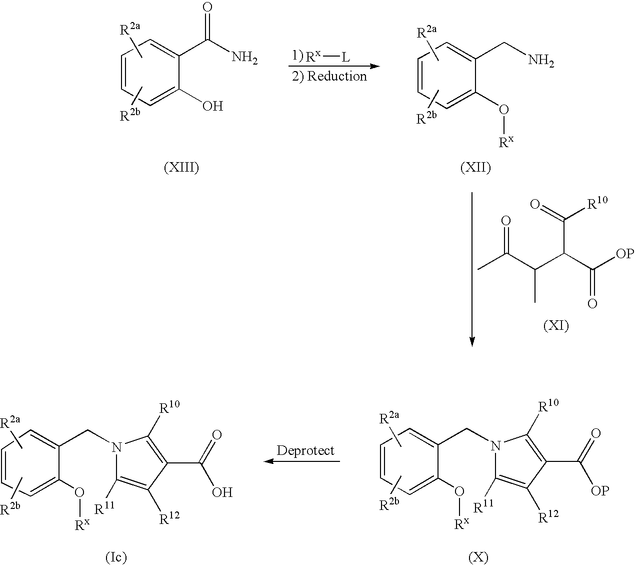

wherein L is a leaving group for example halo, e.g. bromo; P is a protecting group for example C₁₋₄ alkyl e.g. methyl or ethyl; and R^{2a}, R^{2b}, R¹⁰, R¹¹, R¹², and R^{x} are as defined for compounds of formula (Ia).

The skilled person will appreciate that one substituent R^{x} can be converted to a different substituent R^{x} by conventional means, as described, for example, in the methods of the Examples, at a suitable point during the synthesis.

Suitable reaction conditions for the reaction of a compound of formula (XIII) with a compound R^{x}-L are known to those skilled in the art and include the use of a solvent e.g. acetone in the presence of a base, e.g. potassium carbonate.

Suitable conditions for the reduction of the primary amide to give an amine of formula (XIII) are well known and include, for example lithium aluminium hydride in THF.

Suitable reaction conditions for the condensation of (XI) and (XII) to give a pyrrole of formula (X) are known to the skilled person and include ethyl acetate/acetic acid at ambient temperature.

Suitable conditions for the deprotection of an ester (X) to give the corresponding carboxylic acid of formula (1c) are known to those skilled in the art.

Accordingly the present invention also provides a process for the preparation of a compound of formula (Ic) or a derivative thereof: 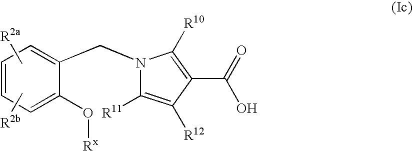
wherein:

R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;

R^{x} represents optionally substituted alkyl wherein 1 or 2 of the non-terminal carbon atoms are optionally substituted by a group independently selected from NR⁴, O and SOₙ, wherein n is 0, 1 or 2: or R^{x} may be optionally substituted CQ^{a}Q^{b}-heterocyclyl, optionally substituted CQ^{a}Q^{b}-bicyclic heterocyclyl or optionally substituted CQ^{a}Q^{b}-aryl;

R⁴ represents hydrogen or an optionally substituted alkyl;

R⁵ represents hydrogen or an optionally substituted alkyl;

R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;

R⁷ represents hydrogen, optionally substituted alkyl, optionally substituted heteroaryl or optionally substituted aryl;

R¹⁰ represents hydrogen, halogen, optionally substituted alkyl, optionally substituted aryl, or optionally substituted heterocyclyl;

R¹¹ represents hydrogen, halogen, optionally substituted alkyl, optionally substituted aryl, or optionally substituted heterocyclyl; and

R¹² represents hydrogen, halogen, CH₃ or CF₃;

wherein Q^{a} and Q^{b} are each independently selected from hydrogen, CH₃ and fluorine;

comprising:

reacting a compound of formula (XII): 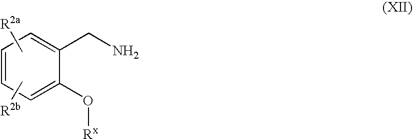
wherein R^{2a}, R^{2b}, and R^{x} are as defined above for a compound of formula (Ib);

with a compound of formula (XI): 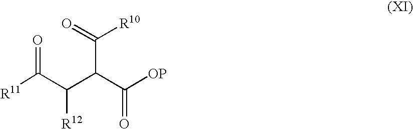
wherein R¹⁰, R¹¹, and R¹² are as defined for compounds of formula (I) and P is a protecting group;

removing the protecting group;

and, if required, forming a derivative thereof.

Compounds R^{x}-L and compounds of formula (III), (V), (VII), (IX) and t-butyl carbazate are commercially available, or may be readily prepared from commercially available intermediates by methods known to those skilled in the art.

Compounds of formula R^{x}-L wherein L is as defined above and R^{x} is as defined for compounds of formula (I) are commercially available, or may be readily prepared by known transformations of commercially available compounds.

Compounds of formula (III):

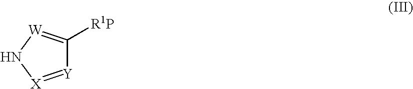

wherein W, X, Y and R¹ are as defined for compounds of formula (I) and P is an optional protecting group are commercially available, or may be prepared by conventional processes for the preparation of pyrroles, pyrazoles, triazoles and tetrazoles. The preparation of pyrroles, pyrazoles, tetrazoles and triazoles is reviewed in e.g. Comprehensive heterocyclic chemistry. The structure, reactions, synthesis and uses of heterocyclic compounds, A. R. Katritzky and C. W. Rees (Eds), vols 1-8, Pergamon Press, Oxford, 1984; Comprehensive organic chemistry II. A review of the literature 1982-1995, A. R. Katritzky, C. W. Rees, and E. F. V. Scriven (eds), vols 1-11, Pergamon Press, Oxford, 1996, and Heterocyclic Chemistry, 4th Edition, J. A. Joule and K. Mills, Blackwell Science, 2000.

Compounds of formula (V): 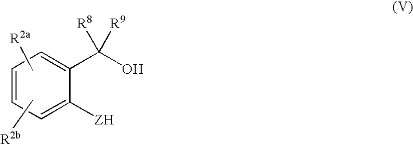
wherein Z, R^{2a}, R^{2b}, R⁸, and R⁹ are as defined for compounds of formula (I) are commercially available, or may be prepared from commercially available intermediates by conventional methods. For example, processes for the preparation of 2-(hydroxymethyl)phenols are described in W. A. Sheppard, J. Org. Chem., 1968, 33, 3297-3306.

Intermediates of formula (VII): 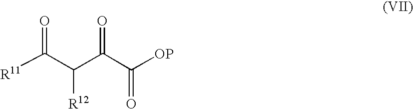
wherein R¹¹ and R¹² are as defined for compounds of formula (Ia), and P is C₁₋₄ alkyl e.g. methyl or ethyl, are commercially available or may be prepared from commercial intermediates by known processes for the preparation of 1,3-diketones e.g. J. Royals, J. Amer. Chem. Soc. 1945, 67, 1508.

Intermediates of formula (IX): 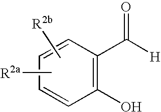
wherein R^{2a} and R^{2b} are as defined for compounds of formula (I) are commercially available, or may readily be prepared by methods known to those skilled in the art, for example from suitable commercially available starting materials using methods as described in the examples. The preparation of aldehydes is reviewed in The Chemistry of the Carbonyl Group, S. Patai (Ed), Interscience, New York, 1966, and references cited therein.

Intermediates of formula (XI): 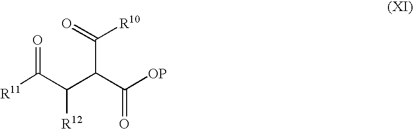
wherein R¹⁰, R¹¹ and R¹² are as defined for compounds of formula (I) are commercially available, or may readily be prepared by methods known to those skilled in the art, from suitable commercially available starting materials using methods as described in the examples.

Intermediates of formula (XIII): 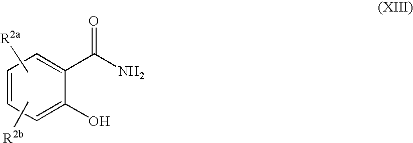
wherein R^{2a} and R^{2b} are as defined for compounds of formula (I) are commercially available, or may readily be prepared by methods known to those skilled in the art, for example from suitable commercially available starting materials. The preparation of benzamides is reviewed in The Chemistry of the Amides, Zabicky (Ed), Interscience, New York, 1970, and references cited therein.

Certain substituents in any of the reaction intermediates and compounds of formula (I) may be converted to other substituents by conventional methods known to those skilled in the art. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4. Fluorination of pyrazoles is described in e.g. K. Makino et al, J. Fluor. Chem, 1988, 39, 435440. When R¹⁰ is alkyl, the R¹⁰ group may be incorporated via C-metallation and alkylation as described in, for example, Heterocyclic Chemistry, 4th Edition, J. A. Joule and K. Mills, Blackwell Science, 2000.

For example, when R^{x} is p-methoxybenzyl, cleavage of the ether to give the phenol is carried out using, for example, using acid e.g. HCl/dioxane or using sodium methanethiolate. Conversion to another R^{x} group, for example a substituted benzyl group, may be effected by reaction of the phenol with a suitable substituted benzyl bromide. The skilled person will appreciate that conversion of the protecting group P to another protecting group P may also occur under the reaction conditions used. When R^{x} is benzyl, cleavage of the ether to give the phenol may be carried out by hydrogenation according to known methods e.g. H₂—Pd/C or NH₄CO₂H—Pd/C. The resulting phenol can then be converted to another group R^{x} as described above.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The compounds of the invention bind to the EP₁ receptor and are therefore considered useful in treating conditions mediated by the action of PGE₂ at EP₁ receptors.

Conditions mediated by the action of PGE₂ at EP₁ receptors include pain; fever; inflammation; immunological diseases; abnormal platelet function diseases; impotence or erectile dysfunction; bone disease; hemodynamic side effects of non-steroidal anti-inflammatory drugs; cardiovascular diseases; neurodegenerative diseases and neurodegeneration; neurodegeneration following trauma; tinnitus; dependence on a dependence-inducing agent; complications of Type I diabetes; and kidney dysfunction.

The compounds of formula (I) are considered to be useful as analgesics. They are therefore considered useful in the treatment or prevention of pain.

The compounds of formula (I) are considered useful as analgesics to treat acute pain, chronic pain, neuropathic pain, inflammatory pain, visceral pain, pain associated with cancer and fibromyalgia, pain associated with migraine, tension headache and duster headaches, and pain associated with functional bowel disorders, non-cardiac chest pain and non-ulcer dispepsia.

The compounds of formula (I) are considered useful in the treatment of chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea. The compounds of the invention may also be considered useful in the treatment of visceral pain.

The compounds of the invention are considered to be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) are also considered useful in the treatment of fever.

The compounds of formula (I) are also considered useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

The compounds of formula (I) are also considered useful in the treatment of immunological diseases such as autoimmune diseases, immunological deficiency diseases or organ transplantation. The compounds of formula (I) are also effective in increasing the latency of HIV infection.

The compounds of formula (I) are also considered useful in the treatment of diseases relating to abnormal platelet function (e.g. occlusive vascular diseases).

The compounds of formula (I) are also considered useful for the preparation of a drug with diuretic action.

The compounds of formula (I) are also considered useful in the treatment of impotence or erectile dysfunction.

The compounds of formula (I) are also considered useful in the treatment of bone disease characterised by abnormal bone metabolism or resorbtion such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

The compounds of formula (I) are also considered useful for attenuating the hemodynamic side effects of non-steroidal anti-inflammatory drugs (NSAID's) and cyclooxygenase-2 (COX-2) inhibitors.

The compounds of formula (I) are also considered useful in the treatment of cardiovascular diseases such as hypertension or myocardiac ischemia; functional or organic venous insufficiency; varicose therapy; haemorrhoids; and shock states associated with a marked drop in arterial pressure (e.g. septic shock).

The compounds of formula (I) are also considered useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

The compounds of formula (I) are also considered useful in the treatment of tinnitus.

The compounds of formula (I) are also considered useful in preventing or reducing dependence on, or preventing or reducing tolerance or reverse tolerance to, a dependence—inducing agent. Examples of dependence inducing agents include opioids (e.g. morphine), CNS depressants (e.g. ethanol), psychostimulants (e.g. cocaine) and nicotine.

The compounds of formula (I) are also considered useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of formula (I) are also considered useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The compounds of formula (I) are also useful in the treatment of overactive bladder and urge incontenance.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof adapted for use in human or veterinary medicine.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands and vanilloid receptor ligands. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO002/18374.

The invention thus provides, in a further aspect, a combination comprising a compound of formula (I) or a pharmaceutically acceptable derivative thereof together with a further therapeutic agent or agents.

In addition to activity at the EP₁ receptor, the compounds of the present invention and pharmaceutically acceptable derivatives thereof exhibit antagonism of the TP receptor and are therefore indicated to be useful in treating conditions mediated by the action of thromboxane at the TP receptor.

In view of their antagonism of the TP receptor, the compounds of the invention and pharmaceutically acceptable derivatives thereof are indicated to be useful in the treatment of renal disorders, asthma, or gastric lesions.

Certain compounds of the invention are equipotent antagonists of the EP₁ and TP receptors.

The present invention therefore also provides a compound which is an equipotent antagonist of the TP receptor and the EP₁ receptor.

According to another aspect of the invention, we provide a compound of formula (I) or a pharmaceutically acceptable derivative thereof for use in the treatment of a condition which is mediated by the action of thromboxane at the TP receptor.

According to a further aspect of the invention, we provide a method of treating a human or animal subject suffering from a condition which is mediated by the action of thromboxane at the TP receptor which comprises administering to said subject an effective amount of a compound of formula (I) or a pharmaceutically acceptable derivative thereof.

According to a yet further aspect of the invention we provide a method of treating a human or animal subject suffering from a renal disorder, asthma, or gastric lesions, which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of thromboxane at the TP receptor.

According to another aspect of the invention we provide the use of a compound of formula (I) or a pharmaceutically acceptable derivative thereof for the manufacture of a medicament for the treatment or prevention of a condition such as a renal disorder, asthma, or gastric lesions.

In certain situations it is envisaged that the administration of a compound exhibiting antagonism of TP receptors in combination with a compound exhibiting antagonism of EP₁ receptors may be advantageous.

The present invention therefore also provides a composition comprising an EP₁ antagonist or a pharmaceutically acceptable derivative thereof and a TP antagonist or a pharmaceutically acceptable derivative thereof.

According to a further aspect, we provide a combination comprising an EP₁ antagonist or a pharmaceutically acceptable derivative thereof and a TP antagonist or a pharmaceutically acceptable derivative thereof for use in the treatment of a condition which is mediated by the action of PGE₂ at EP₁ receptors.

The present invention also provides a combination comprising an EP₁ antagonist or a pharmaceutically acceptable derivative thereof and a TP antagonist or a pharmaceutically acceptable derivative thereof for use in the treatment of pain, or inflammatory, immunological, bone, neurodegenerative or renal disorders.

The present invention further provides a combination comprising an EP₁ antagonist or a pharmaceutically acceptable derivative thereof and a TP antagonist or a pharmaceutically acceptable derivative thereof for use in the treatment of inflammatory pain, neuropathic pain or visceral pain.

According to a further aspect of the invention we provide a method of treating a human or animal subject suffering from a pain, or an inflammatory, immunological, bone, neurodegenerative or renal disorder, which method comprises administering to said subject a combination comprising an effective amount of an EP₁ antagonist or a pharmaceutically acceptable derivative thereof and an effective amount of a TP antagonist or a pharmaceutically acceptable derivative thereof.

According to a yet further aspect of the invention we provide a method of treating a human or animal subject suffering from inflammatory pain, neuropathic pain or visceral pain which method comprises administering to said subject a combination comprising an effective amount of an EP₁ antagonist or a pharmaceutically acceptable derivative thereof and an effective amount of a TP antagonist or a pharmaceutically acceptable derivative thereof.

According to another aspect of the invention, we provide the use an EP₁ antagonist or a pharmaceutically acceptable derivative thereof in combination with a TP antagonist or a pharmaceutically acceptable derivative thereof for the manufacture of a medicament for the treatment of a condition which is mediated by the action of PGE₂ at EP₁ receptors.

According to yet another aspect of the invention we provide the use an EP₁ antagonist or a pharmaceutically acceptable derivative thereof in combination with a TP antagonist or a pharmaceutically acceptable derivative thereof for the manufacture of a medicament for the treatment of or prevention of a condition such as a pain, or an inflammatory, immunological, bone, neurodegenerative or renal disorder.

According to a further aspect of the invention we provide the use of use an EP₁ antagonist or a pharmaceutically acceptable derivative thereof in combination with a TP antagonist or a pharmaceutically acceptable derivative thereof for the manufacture of a medicament for the treatment or prevention of a condition such as inflammatory pain, neuropathic pain or visceral pain.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A proposed daily dosage of compounds of formula (I) or their pharmaceutically acceptable derivatives for the treatment of man is from 0.01 to 30 mg/kg body weight per day and more particularly 0.1 to 10 mg/kg body weight per day, which may be administered as a single or divided dose, for example one to four times per day The dose range for adult human beings is generally from 8 to 2000 mg/day, such as from 20 to 1000 mg/day, preferably 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

No unacceptable toxicological effects are expected with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

Abbreviations

Bn (benzyl), Bu, Pr, Me, Et (butyl, propyl, methyl ethyl), DMSO (dimethyl sulfoxide), DCM (dichloromethane), EDTA (ethylenediamine tetraacetic acid), EtOAc (ethyl acetate), EtOH (ethanol), HPLC (High pressure liquid chromatography), LCMS (Liquid chromatography/Mass spectroscopy), MDAP (Mass Directed Purification), MeCN (acetonitrile), MeOH (methanol), NMR (Nuclear Magnetic Resonance (spectrum)), Ph (phenyl), SPE (Solid Phase Extraction), THF (tetrahydrofuran), s, d, t, q, m, br (singlet, doublet, triplet, quartet, multiplet, broad.)

LCMS
Column: 3.3 cm×4.6 mm ID, 3 um ABZ+PLUS
Flow Rate: 3 ml/min
Injection Volume: 5 μl
Temp: RT
UV Detection Range: 215 to 330 nm

Solvents: A: 0.1% Formic Acid+10 mMolar Ammonium Acetate.

B: 95% Acetonitrile+0.05% Formic Acid

| Gradient: | Time | A % | B % |
|---|---|---|---|
| | 0.00 | 100 | 0 |
| | 0.70 | 100 | 0 |
| | 4.20 | 0 | 100 |
| | 5.30 | 0 | 100 |
| | 5.50 | 100 | 0 |

Mass Directed Autopreparation

Hardware:

Waters 600 gradient pump

Waters 2767 inject/collector

Waters Reagent Manager

Micromass ZMD mass spectrometer

Gilson Aspec—waste collector

Gilson 115 post-fraction UV detector

Software:

Micromass Masslynx version 4.0

Column

The column used is typically a Supelco LCABZ++ column whose dimensions are 20 mm internal diameter by 100 mm in length. The stationary phase particle size is 5 μm.

Solvents:

A:. Aqueous solvent=Water+0.1% Formic Acid

B: Organic solvent=MeCN: Water 95:5+0.05% Formic Acid

Make up solvent=MeOH: Water 80:20+50 mMol Ammonium Acetate

Needle rinse solvent=MeOH:Water:DMSO 80:10:10

The method used depends on the analytical retention time of the compound of interest. 15-minute runtime, which comprises a 10-minute gradient followed by a 5-minute column flush and re-equilibration step.

MDP 1.5-2.2=0-30% B

MDP 2.0-2.8=5-30% B

MDP 2.5-3.0=15-55% B

MDP 2.8-4.0=30-80% B

MDP 3.8-5.5=50-90% B

Flow rate:

flow rate 20 ml/min.

### General Method1

### Preparation of 4-bromo-2-(bromomethyl)phenyl phenylmethyl ether (Intermediate A)

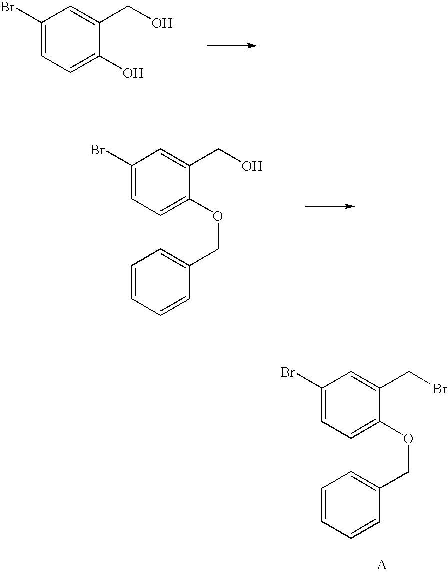

### a) {5-bromo-2-[(phenylmethyl)oxy]phenyl}methanol

4-bromo-2-(hydroxymethyl)phenol (10.15 g, 50 mmol) was dissolved in ethanol (100 ml) and 2M sodium hydroxide (27.5 ml, 55 mmol). The resulting solution was stirred for 10 minutes. A solution of benzyl bromide (5.95 ml, 50 mmol) in ethanol (100 ml) was added slowly and the resulting solution was stirred overnight at room temperature. The reaction mixture was concentrated in vacuo, the solution obtained diluted with water and extracted with dichloromethane. The combined organic layers were washed sequentially with a saturated solution of NaHCO₃ and water, dried (Na₂SO₄) filtered and evaporated to dryness. The residue was purified by flash chromatography using dichloromethane to yield the title compound as a colourless oil (13.8 g, 94%).

¹H NMR δ: 2.19 (1H, t), 4.71 (2H, d, J=6.3 Hz), 5.10 (2H, s), 6.82 (1H, d, J=8.6 Hz), 7.34-7.47 (7H, m).

### b) 4-bromo-2-(bromomethyl)phenyl phenylmethyl ether (Intermediate A)

A solution of {5-bromo-2-[(phenylmethyl)oxy]phenyl}methanol (5.41 g, 18.44 mmol) in dichloromethane (30 ml) was stirred under nitrogen and cooled to −10° C. (ice/acetone). A solution of phosphorous tribromide (4.99 g, 1.75 ml,18.44 mmol) in dichloromethane (15 ml) was added slowly at −10° C. and the mixture warmed to −7° C. and stirred for 15 mins. The reaction was then allowed to warm to room temperature and was stirred overnight under nitrogen. The reaction mixture was cooled (ice/water bath) and a saturated sodium hydrogen carbonate solution (15.5 ml) was then added slowly and the mixture diluted with dichloromethane and water. The organic phase was separated, washed with water then dried (Na₂SO₄) and evaporated to dryness. The residue was purified by flash chromatography with diethyl ether to yield the title compound as a white solid (5.53 g, 84%).

¹H NMR δ: 4.54 (2H, s), 5.15 (2H, s), 6.81 (1H, d, J=8.8 Hz), 7.33-7.48 (7H, m)

The following example was prepared using General Method 1 (b) from {5-chloro-2-[(phenylmethyl)oxy]phenyl}methanol.

### 4-chloro-2-(bromomethyl)phenyl phenylmethyl ether

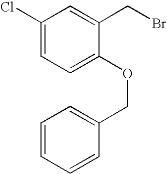

t=3.27, no ion observed.

### General Method2

### Example 1

### 1-({5-bromo-2-[(phenylmethyl)oxy]phenyl}methyl)-5-methyl-1H-pyrazole-3-carboxylic acid

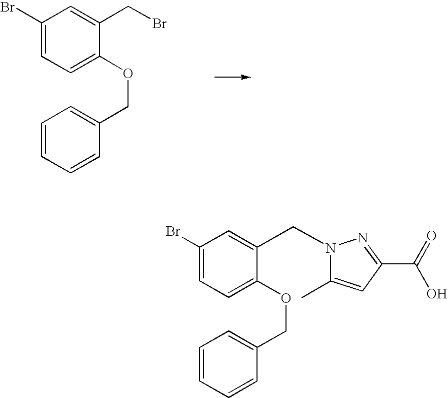

Methyl 1H-pyrazole-3-carboxylate (12.61 mg, 0.1 mmol) was dissolved in a 0.105M solution of potassium tert-butoxide in ethanol (1 ml, 11.78 mg, 0.1 05 mmol). After stirring at room temperature for 5 mins, a 0.1M solution of 4-bromo-2-(bromomethyl)phenyl phenylmethyl ether in ethanol (1 ml, 35.6 mg, 0.1 mmol) was added and the resulting solution was stirred and heated at 60° C. under nitrogen for 4 hrs. After cooling the mixture was diluted with ethanol (1 ml) and a 0.5M solution of lithium hydroxide in water (1 ml, 11.97 mg, 0.5 mmol) was added. The mixture was stirred overnight at 40° C. After cooling 2M hydrochloric acid (0.3 ml, 0.6 mmol) was added and the mixture was diluted with water. Dichloromethane was added and the mixture stirred vigorously. The organic layer was separated and the solvent removed in vacuo. The residue was purified by mass directed autopurification to yield the title compound. 1-({5-bromo-2-[(phenylmethyl)oxy]phenyl}methyl)-5-methyl-1H-pyrazole-3-carboxylic acid: (10.7 mg, 27.6%).

¹H NMR δ: 5.08 (2H, s), 5.34 (2H, s), 6.72 (1H, d, J=2.2 Hz), 6.92 (1H, d, J=8.8 Hz), 7.23 (1H, d, J=2 Hz), 7.30-7.39 (6H, m), 7.45 (1H, d, J=2 Hz).

t=3.38, [MH+] 387, 389 [MH−] 385, 387.

### 1-({5-chloro-2-[(phenylmethyl)oxy]phenyl}methyl)-1H-pyrazole-3-carboxylic acid ethyl ester

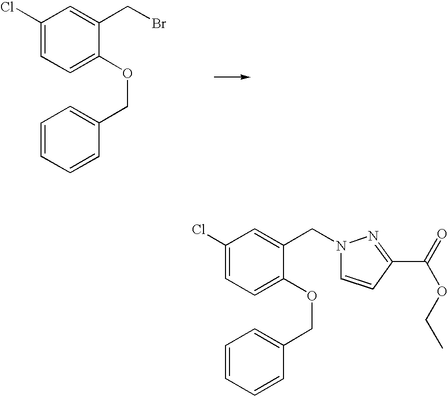

1H-pyrazole-3-carboxylic add ethyl ester (13.0 g, 93 mmol) was dissolved in dimethylformamide (200 ml). Potassium carbonate (32 g, 232 mmol) was added to the solution, followed by 4-chloro-2-(bromomethyl)phenyl phenylmethyl ether (29 g, 93 mmol) and the reaction mixture stirred overnight at room temperature under argon. Water and ethyl acetate were added and the layers separated. The aqueous phase was re-extracted with ethyl acetate. The organic phases were combined and washed with water followed by brine. The extracts were dried (Na₂SO₄) and evaporated. The residue was purified by on a biotage (15-25% ethyl acetate:hexane) to yield the title compounds.

1-({5-chloro-2-[(phenylmethyl)oxy]phenyl}methyl)-1H-pyrazole-3-carboxylic acid ethyl ester: (13.90 g, 40%).

t=3.40, no ion observed.

### Example 2

### 1-({5-chloro-2-[(phenylmethyl)oxy]phenyl}methyl)-1H-pyrazole-3-carboxylic acid

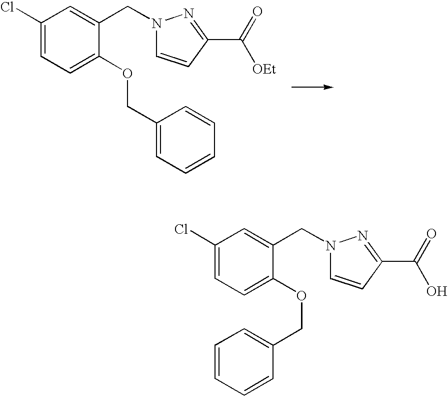

1-({5-chloro-2-[(phenylmethyl)oxy]phenyl}methyl)-1H-pyrazole-3-carboxylic acid ethyl ester (13.9 g, 37.5 mmol) was dissolved in ethanol (150 ml) and 2M sodium hydroxide (45 ml, 90 mmol). This mixture was stirred at reflux for 4 hours. The ethanol was evaporated and the mixture diluted with ethyl acetate and water. This was acidified with 2M hydrochloric acid, and the phases separated. The aqueous phase was re-extracted with ethyl acetate, the organic layers combined, dried (Na₂SO₄) and evaporated to dryness, to give the title compound as a white solid (12.09 g, 94%).

t=2.98, [MH−] 341.

### General Method 3

### Preparation of ethyl 1-[(5-bromo-2-hydroxyphenyl)methyl]-5-methyl-1H-pyrazole-3-carboxylate (Intermediate B)

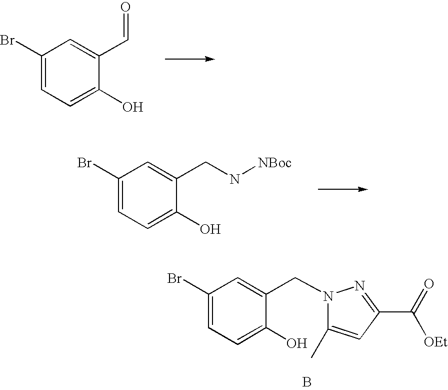

### a) 1,1-dimethylethyl 2-[(5-bromo-2-hydroxyphenyl)methyl]hydrazinecarboxylate

5-bromo-2-hydroxybenzaldehyde (4.02 g, 20 mmol) was dissolved in dichloromethane (100 ml). Tert-butyl carbazate (2.64 g, 20 mmol) and acetic acid (1.14 ml, 1.2 g, 20 mmol) were added and the mixture was stirred under nitrogen for 30 mins. Sodium triacetoxyborohydride (12.72 g, 60 mmol) was added portionwise and the resulting suspension was then stirred overnight under nitrogen. 2M hydrochloric acid (30 ml, 60 mmol) was added and the resulting solution was diluted with dichloromethane and water. The organic phase was separated, washed sequentially with brine and water then dried (Na₂SO₄) and evaporated to dryness to give the title compound as a white solid (6.01 g, 94.7%)

¹H NMR δ: 1.48 (9H, s), 4.13 (2H, s), 4.40 (1H, br s), 6.15 (1H, br s), 6.78 (1H, d, J=8.8 Hz), 7.16 (1H, d, J=2.26 Hz), 7.29-7.32 (1H, m), 9.28 (1H, br s).

t=3.11, [MH+] 317, 319 [MH−] 315, 317.

### b) Ethyl 1-[(5-bromo-2-hydroxyphenyl)methyl]-5-methyl-1H-pyrazole-3-carboxylate (Intermediate B)

Trifluoroacetic acid (20 ml) was added to 1,1-dimethylethyl 2-[(5-bromo-2-hydroxyphenyl)methyl]hydrazinecarboxylate (3.2 g, 10 mmol) in dichloromethane (40 ml) and the reaction mixture stirred overnight at room temperature under nitrogen. The solvent was removed in vacuo and the residue obtained redissolved in acetic acid (20 ml). The resulting solution was added dropwise to a solution of ethyl 2,4-dioxopentanoate (1.40 ml, 1.58 g, 10 mmol) in acetic acid (10 ml) and the reaction mixture was heated at reflux under nitrogen for 1 h. The title compound crystallized upon cooling, was filtered, washed with acetic acid and dried under vacuo to give the title compound as white crystals (1.85 g, 54.7%)

¹H NMR δ: 1.39 (3H, t, J=7.15 Hz), 2.41 (3H, s), 4.34-4.40 (2H, q), 5.18 (2H, s), 6.58 (1H, s), 6.88 (1H, d, J=8.5 Hz), 7.24 (1H, d, J=2.2 Hz), 7.33 (1H, m), 9.56 (1H, br s).

t=3.17, [MH+] 339, 341 [MH−] 337, 339.

### General Method4

### Example 3

### 1-[(5-Bromo-2-{[(2,4-difluorophenyl)methyl]oxy}phenyl)methyl]-5-methyl-1H-pyrazole-3-carboxylic acid

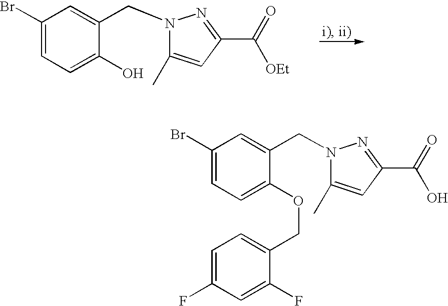

Ethyl 1-[(5-bromo-2-hydroxyphenyl)methyl]-5-methyl-1H-pyrazole-3-carboxylate (16.95 mg, 0.05 mmol) was dissolved in ethanol (0.5 ml) and 2M sodium hydroxide (0.0275 ml, 0.055 mmol) and stirred at room temperature for 5 mins. 1-(bromomethyl)-2,4-difluorobenzene (10.35 mg, 0.05 mmol) in ethanol (0.5 ml) was added and the reaction mixture heated under nitrogen at 70° C. overnight. After cooling the mixture was diluted with ethanol (0.5 ml) and a 0.5M solution of lithium hydroxide in water (0.5 ml, 5.99 mg, 0.25 mmol) was added. The mixture was stirred at 40° C. for 3 h. After cooling 2M hydrochloric acid (0.15 ml, 0.3 mmol) and the mixture was diluted with water. Dichloromethane was added and the mixture stirred vigorously. The organic layer was separated and the solvent removed in vacuo. The residue was purified by mass directed autopurification to yield the title compound (18.4 mg, 84.2%).

¹H NMR δ: 2.13(3H, s), 5.10 (2H, s), 5.27 (2H, s), 6.55 (1H, s), 6.85 (1H, d, J=2 Hz), 6.89-6.97 (3H, m), 7.36-7.46 (2H, m).

t=3.48, [MH+] 437, 439 [MH−] 435, 437.

### Ethyl 1-({5-chloro-2-[(2-methylpropyl)oxy]phenyl}methyl)-5-methyl-1H-pyrazole-3-carboxylate

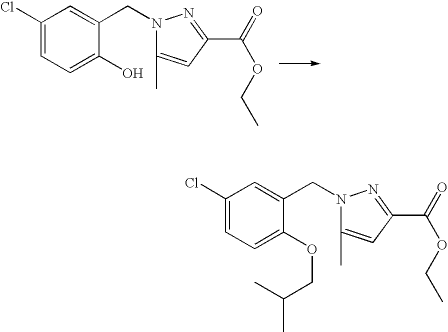

A mixture of ethyl 1-[(5-chloro-2-hydroxyphenyl)methyl]-5-methyl-1H-pyrazole-3-carboxylate (25.82 g, 0.088 mol), potassium carbonate (0.176 mol, 24.3 g), isobutyl bromide (0.132 mol, 14.2 ml) was stirred in dimethylformamide (175 ml) at 116° C. for 16 hours. After this time further isobutyl bromide (0.044 mol, 4.7 ml) was added and the reaction continued for 2 hours. This was evaporated to a solid and then partitioned between ethyl acetate (500 ml) and water (200 ml). The aqueous was run off and the organic washed twice with water (100 ml) and once with brine (50 ml) before being evaporated to a solid which was flash chromatographed with hexane/ethyl acetate (4/1) to give the title compound (29.12 g) LC/MS [M+H] 351 and 353, Rt=3.47 min

### Example 4

### Sodium 1-({5-chloro-2-[(2-methylpropyl)oxy]phenyl}methyl)-5-methyl-1H-pyrazole-3-carboxylate

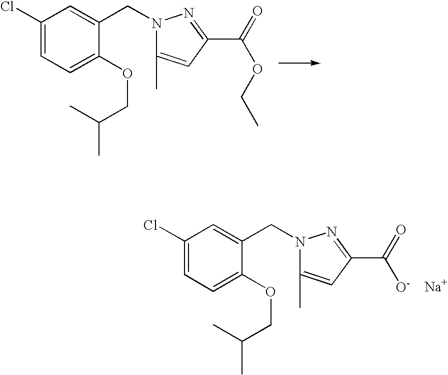

Ethyl 1-({5-chloro-2-[(2-methylpropyl)oxy]phenyl}methyl)-5-methyl-1H-pyrazole-3-carboxylate (0.083 mol, 29.12 g) was stirred in ethanol (330 ml) and 2N sodium hydroxide (100 ml) at 95° C. for 1½ hours. This was cooled to room temperature and evaporated to a solid. This was partitioned between EtOAc (500 ml) and water (200 ml). Some of the ethyl acetate layer (5 ml) was taken, washed with brine (2 ml) and dried over magnesium sulphate and evaporated to give the title compound (0.225 g). LC/MS [M+Na] 345 and 347, Rt=2.94 min

Regioisomers: Elucidation of isolated structures where regioisomers can be formed (general methods 2 and 3) was determined by using either NMBC (heteronuclear multiple bond correlation); nOe (nuclear Overhauser effect) NMR techniques.

The following Examples were prepared from either Intermediate A or Intermediate B and Methods 2 or 4, and other appropriate starting materials.

| | | | |
|---|---|---|---|
| 5 | 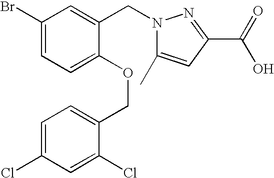 | Name NMR LCMS Method | 1-{5-bromo-2-[(2,4-dichlorobenzyl)oxy]benzyl}-5-meth- yl-1H-pyrazole-3-carboxylic acid ¹H NMR δ: 2.15(3H, s), 5.13(2H, s), 5.31(2H, s), 6.57(1H, s), 6.80(1H, d, J=2.5Hz), 6.86(1H, d, J=8.8Hz), |
| #7.28(1H, m), 7.35(1H, m), 7.39(1H, d, J=8.3Hz), 7.43(1H, d, J=2Hz) t = 3.84, [MH+] 467, 469, 471 [MH−] 469, 471, 473 B and Method 4 | | | |
| 6 | 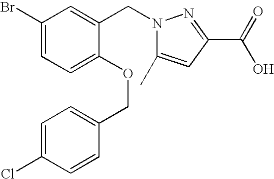 | Name NMR LCMS Method | 1-{5-bromo-2-[(4-chlorobenzyl)oxy]benzyl}-5-meth- yl-1H-pyrazole-3-carboxylic acid ¹H NMR δ: 2.16(3H, s), 5.10(2H, s), 5.32(2H, s), 6.57(1H, s), 6.91(1H, d, J=2.2Hz), 6.99(1H, d, J=8.8Hz), 7.38-7.40(5H, |
| #m) t = 3.60, [MH+] 435, 437 [MH−] 433, 435 B and Method 4 | | | |
| 7 | 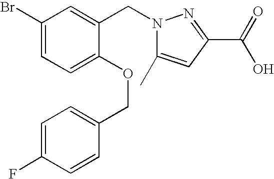 | Name NMR LCMS Method | 1-{5-bromo-2-[(4-fluorobenzyl)oxy]benzyl}-5-meth- yl-1H-pyrazole-3-carboxylic acid ¹H NMR δ: 2.14(3H, s), 5.09(2H, s), 5.30(2H, s), 6.57(1H, s), 6.92(1H, d, J=2Hz), 7.00(1H, d, J=8.8Hz), 7.10(2H, |
| #t, J=8.8Hz), 7.38-7.44(3H, m) t = 3.44, [MH+] 419, 421 [MH−] 417, 419 B and Method 4 | | | |
| 8 | 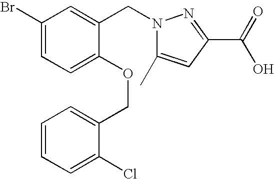 | Name NMR LCMS Method | 1-{5-bromo-2-[(2-chlorobenzyl)oxy]benzyl}-5-meth- yl-1H-pyrazole-3-carboxylic acid ¹H NMR δ: 2.13(3H, s), 5.17(2H, s), 5.32(2H, s), 6.56(1H, s), 6.80(1H, d, J=2.3Hz), 6.88(1H, d, J=8.8Hz), 7.25-7.43(5H, |
| #m) t = 3.66, [MH+] 435, 437 [MH−] 433, 435 B and Generic Method 4 | | | |
| 9 | 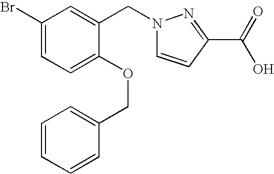 | Name NMR LCMS Method | 1-[2-(benzyloxy)-5-bromobenzyl]-1H-pyrazole-3-car- boxylic acid ¹H NMR δ: 5.08(2H, s), 5.34(2H, s), 6.72(1H, d, J=2.2Hz), 6.92(1H, d, J=8.8Hz), 7.23(1H, d, J=2Hz), 7.30-7.39(6H, m), 7.45(1H, d, J=2Hz) t = 3.38, |
| #[MH+] 487, 489 [MH−] 485, 487 A and Method 2 | | | |
| 10 | 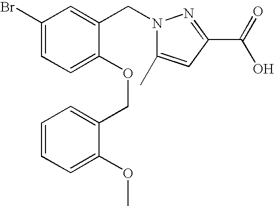 | Name NMR LCMS Method | 1-{5-bromo-2-[(2-methoxybenzyl)oxy]benzyl}-5-meth- yl-1H-pyrazole-3-carboxylic acid ¹H NMR δ: 2.12(3H, s), 3.83(3H, s), 5.10(2H, s), 5.28(2H, s), 6.54(1H, s), 6.85(1H, d), 6.91-6.96(3H, m), 7.28-7.35(3H, |
| #m) t = 3.52, [MH+] 431, 433 [MH−] 429, 431 B and Method 4 | | | |
| 11 | 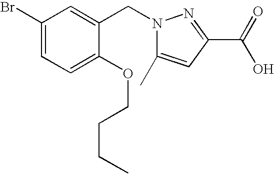 | Name NMR LCMS Method | 1-(5-bromo-2-butoxybenzyl)-5-methyl-1H-py- razole-3-carboxylic acid ¹H NMR δ: 0.96(3H, t, J=7.4Hz), 1.43-1.52(2H, m), 1.73-1.80(2H, m), 2.21(3H, s), 3.99(2H, t, J=6.4Hz), 5.28(2H, |
| #s), 6.58(1H, s), 6.70(1H, d, J=2.2Hz), 6.78(1H, d, J=8.8Hz), 7.29-7.32(1H, m) t = 3.55, [MH+] 367, 369 [MH−] 365, 367 B and Method 4 | | | |
| 12 | 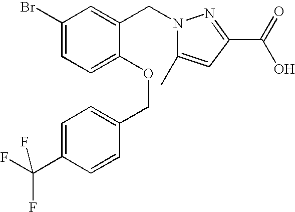 | Name NMR LCMS Method | 1-(5-bromo-2-{[4-(tri- fluoromethyl)benzyl]oxy}benzyl)-5-methyl-1H-py- razole-3-carboxylic acid ¹H NMR δ: 2.18(3H, s), 5.23(2H, s), 5.36(2H, s), 6.58(1H, s), 6.91(1H, d, |
| #J=2.2Hz), 7.00(1H, d, J=8.8Hz), 7.38-7.41(1H, m), 7.59(2H, d, J=8.3Hz), 7.68(2H, d, J=8.3Hz) t = 3.61, [MH+] 469, 471, [MH−] 467, 469 B and Method 4 | | | |
| 13 | 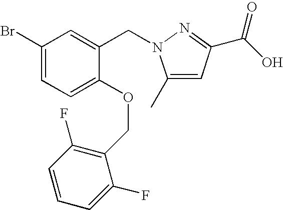 | Name NMR LCMS Method | 1-{-bromo-2-[(2,6-difluorobenzyl)oxy]benzyl}-5-meth- yl-1H-pyrazole-3-carboxylic acid ¹H NMR δ: 2.08(3H, s), 5.18(2H, s), 5.21(2H, s), 6.50(1H, s), 6.94(1H, d, J=2Hz), 7.04(2H, t, J=8Hz), 7.14(1H, d, |
| #J=8.8Hz), 7.43-7.50(2H, m) t = 3.43, [MH+] 437, 439 [MH−] 435, 437 B and Method 4 | | | |
| 14 | 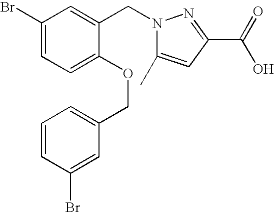 | Name NMR LCMS Method | 1-{5-bromo-2-[(3-bromobenzyl)oxy]benzyl}-5-meth- yl-1H-pyrazole-3-carboxylic acid ¹H NMR δ: 2.16(3H, s), 5.08(2H, s), 5.31(2H, s), 6.58(1H, s), 6.85(1H, d, J=2.1Hz), 6.89(1H, d, J=8.8Hz), 7.24-7.50(5H, |
| #m) t = 3.64, [MH+] 479, 481, 483 [MH−] 477, 479, 481 B and Method 4 | | | |
| 15 | 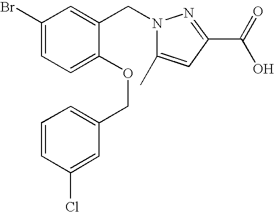 | Name NMR LCMS Method | 1-{5-bromo-2-[(3-chlorobenzyl)oxy]benzyl}-5-meth- yl-1H-pyrazole-3-carboxylic acid ¹H NMR δ: 2.16(3H, s), 5.08(2H, s), 5.31(2H, s), 6.58(1H, s), 6.85(1H, d, J=2Hz), 6.89(1H, d, J=8.8Hz), 7.27-7.36(5H, |
| #m) t = 3.59, [MH+] 435, 437 [MH−] 433, 435 B and Method 4 | | | |
| 16 | 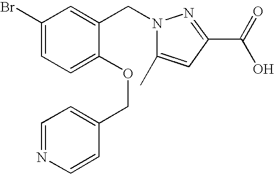 | Name NMR LCMS Method | 1-[5-bromo-2-(pyridin-4-ylmethoxy)benzyl]-5-meth- yl-1H-pyrazole-3-carboxylic acid ¹H NMR δ: 2.23(3H, s), 5.22(2H, s), 5.40(2H, s), 6.61(1H, s), 6.87-6.89(2H, m), 7.35-7.38(1H, m), |
| #7.51(2H, d, J=5.27Hz), 8.53(2H, d, J=4.52Hz) t = 2.58, [MH+] 402, 404 [MH−] 400, 402 B and Method 4 | | | |
| 17 | 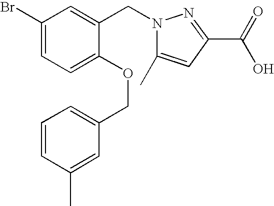 | Name NMR LCMS Method | 1-{5-bromo-2-[(3-methylbenzyl)oxy]benzyl}-5-meth- yl-1H-pyrazole-3-carboxylic acid ¹H NMR δ: 2.12(3H, s), 2.33(3H, s), 5.03(2H, s), 5.29(2H, s), 6.56(1H, s), 6.80(1H, d, J=2Hz), 6.85(1H, |
| #d, J=8.8Hz), 7.11-7.25(4H, m), 7.30-7.33(1H, m) t = 3.58, [MH+] 415, 417 [MH−] 413, 415 B and Method 4 | | | |
| 18 | 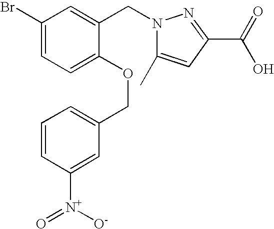 | Name NMR LCMS Method | 1-{5-bromo-2-[(3-nitrobenzyl)oxy]benzyl}-5-meth- yl-1H-pyrazole-3-carboxylic acid t = 3.39, [MH+] 446, 448 [MH−] 444, 446 B and Method 4 |
| 19 | 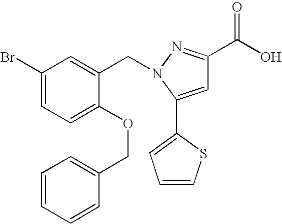 | Name NMR LCMS Method | 1-[2-(benzyloxy)-5-bromobenzyl]-5-thien-2-yl-1H-py- razole-3-carboxylic acid ¹H NMR δ: 5.06(2H, s), 5.50(2H, s), 6.74(1H, d, J=1.8Hz), 6.88(1H, d, J=8.8Hz), 6.95(1H, s), 7.00-7.02(2H, |
| #m), 7.27-7.34(6H, m), 7.43(1H, d, J=5.0Hz) t = 3.83, [MH+] 469, 471 [MH−] 467, 469 A and Method 2 | | | |
| 20 | 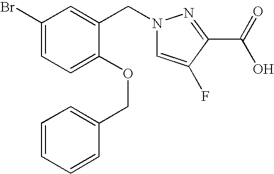 | Name NMR LCMS Method | 1-[2-(benzyloxy)-5-bromobenzyl]-4-fluoro-1H-py- razole-3-carboxylic acid ¹H NMR δ: 5.07(2H, s), 5.23(2H, s), 6.90(1H, d, J=8.8Hz), 7.29-7.40(8H, m) t = 3.48, [MH+] 405, 407 [MH−] 403, 405 A and Method 2 |

The following intermediates were prepared from the appropriate starting materials according to Method 3.

| | | | |
|---|---|---|---|
| C | 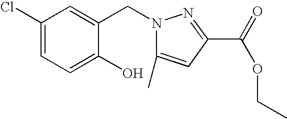 | Name NMR LCMS Method | ethyl 1-[(5-chloro-2-hydroxyphenyl)methyl]-5-meth- yl-1H-pyrazole-3-carboxylate ¹H NMR δ: 1.26(3H, t, J=6.9Hz), 2.27(3H, s), 4.24(2H, q, J=6.9Hz), 5.23(2H, s), 6.58(1H, s), 6.64(1H, s), 6.86(1H, d, J=8.5Hz), |
| #7.17(1H, d, J=8.5Hz), 10.18(1H, s) t = 3.10, [MH+] 295, 297, [MH−] 295, 293 Method 3 | | | |
| D | 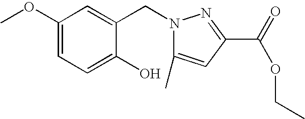 | Name NMR LCMS Method | ethyl 1-{[2-hydroxy-5-(methyloxy)phenyl]methyl}-5-meth- yl-1H-pyrazole-3-carboxylate ¹H NMR δ: 1.39(3H, t, J=7.2Hz), 2.40(3H, s), 3.75(3H, s), 4.37(2H, q, J=6.9Hz), 5.21(2H, s), 6.57(1H, s), |
| #6.70(1H, d, J=3.0Hz), 6.81(1H, dd, J=3.0 and 8.9Hz), 7.93(1H, d, J=8.9Hz), 8.65-8.78(1H, br s) t = 2.83, [MH+] 291, [MH−] 289 Method 3 | | | |
| E | 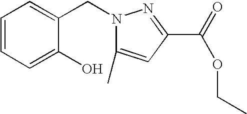 | Name NMR LCMS Method | ethyl 1-[(2-hydroxyphenyl)methyl]-5-methyl-1H-py- razole-3-carboxylate ¹H NMR δ: 1.39(3H, t, J=7.2Hz), 2.40(3H, s), 4.37(2H, q, J=7.2Hz), 5.25(2H, s), 6.57(1H, s), 6.85-6.89(1H, m), 6.99(1H, |
| #dd, J=7.3 and 1.0Hz), 7.14(1H, dd, J=7.5 and 1.5Hz), 7.24(1H, dd, J=1.8 and 8.0Hz), 9.22(1H, s) t = 2.85, [MH+] 261 Method 3 | | | |
| F | 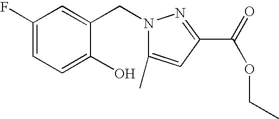 | Name NMR LCMS Method | ethyl 1-[(5-fluoro-2-hydroxyphenyl)methyl]-5-meth- yl-1H-pyrazole-3-carboxylate ¹H NMR δ: 1.39(3H, t, J=7.2Hz), 2.40(3H, s), 4.37(2H, q, J=7.2Hz), 5.20(2H, s), 6.59(1H, s), 6.82(dd, 1H, J=2.0 and 7.5Hz), |
| #6.93(2H, m) t = 2.92, [MH+] 279, 280 Method 3 | | | |

The following Examples were prepared from either Intermediate C, D, E or F according to Method 4.

| | | | |
|---|---|---|---|
| 21 | 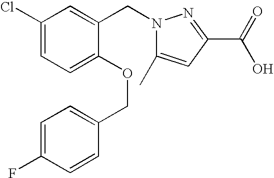 | Name NMR LCMS Method | 1-[(5-chloro-2-{[(4-fluoro- phenyl)methyl]oxy}phenyl)methyl]-5-methyl-1H-py- razole-3-carboxylic acid ¹H NMR δ: 2.16(3H, s), 5.04(2H, s), 5.33(2H, s), 6.67(1H, s), 6.71(1H, d, |
| #J=2.5Hz), 6.87(1H, d, J=8.8Hz), 7.10(2H, t, J=8.8Hz), 7.22(1H, dd, J=8.8 and 2.5Hz), 7.36(2H, dd, J=5.3 and 3.0Hz) t=3.37, [MH+] 375, 377, [MH−] 373, 375 C and Method 4 | | | |
| 22 | 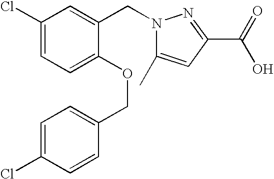 | Name NMR LCMS Method | 1-[(5-chloro-2-{[(4-chloro- phenyl)methyl]oxy}phenyl)methyl]-5-methyl-1H-py- razole-3-carboxylic acid ¹H NMR δ: 2.15(3H, s), 5.10(2H, s), 5.31(2H, s), 6.57(1H, s), 6.76(1H, d, |
| #J=2.5Hz), 7.03(1H, d, J=8.8Hz), 7.24(1H, dd, J=8.8 and 2.5Hz), 7.35-7.40(4H, m) t = 3.51, [MH+] 391, 393, [MH−] 389, 391 C and Method 4 | | | |
| 23 | 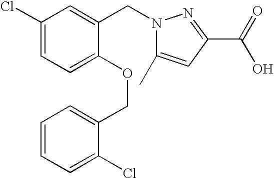 | Name NMR LCMS Method | 1-[(5-chloro-2-{[2-(chloro- phenyl)methyl]oxy}phenyl)methyl]-5-methyl-1H-py- razole-3-carboxylic acid ¹H NMR δ: 2.19(3H, s), 5.19(2H, s), 5.40(2H, s), 6.67(1H, s), 6.79(1H, d, |
| #J=2.3Hz), 6.89(1H, d, J=8.8Hz), 7.22(1H, dd, J=8.8 and 2.3Hz), 7.30-7.32(2H, m), 7.43-7.45(2H, m), t = 3.53, [MH+] 391, 393, [MH−] 389, 391 C and Method 4 | | | |
| 24 | 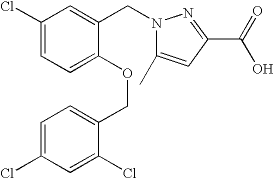 | Name NMR LCMS Method | 1-[(5-chloro-2-{[(2,4-di- chlorophenyl)methyl]oxy}phenyl)methyl]-5-meth- yl-1H-pyrazole-3-carboxylic acid ¹H NMR δ: 2.13(3H, s), 5.20(2H, s), 5.27(2H, s), 6.48(1H, s), |
| #6.85(1H, d, J=2.5Hz), 7.16(1H, d, J=8.8Hz), 7.38(1H, dd, J=8.8 and 2.5Hz), 7.45(1H, dd, J=8.3 and 2.0Hz), 7.62(1H, d, J=8.3Hz), 7.71(1H, d, J=2.3Hz_ t = 3.75, [MH+] 425, 427, 429, [MH−] 423, 425, 427 C and Method 4 | | | |
| 25 | 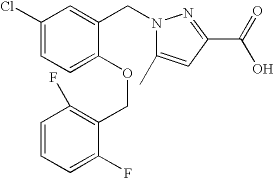 | Name NMR LCMS Method | 1-[(5-chloro-2-{[(2,6-di- fluorophenyl)methyl]oxy}phenyl)methyl]-5-meth- yl-1H-pyrazole-3-carboxylic acid ¹H NMR δ: 2.13(3H, s), 5.17(2H, s), 5.27(2H, s), 6.63(1H, |
| #s), 6.73(1H, d, J=2.5Hz), 6.95-7.02(3H, m), 7.25(1H, d, J=2.5Hz), 7.34-7.41(1H, m) t = 3.36, [MH+] 393, 395, [MH−] 391, 393 C and Method 4 | | | |
| 26 | 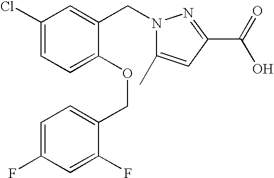 | Name NMR LCMS Method | 1-[(5-chloro-2-{[(2,4-di- fluorophenyl)methyl]oxy}phenyl)methyl]-5-meth- yl-1H-pyrazole-3-carboxylic acid ¹H NMR δ: 2.17(3H, s), 5.09(2H, s), 5.33(2H, s), 6.66(1H, s), |
| #6.71(1H, d, J=2.5Hz), 6.86-6.94(3H, m), 7.24(1H, dd, J=8.8 and 2.5Hz), 7.36-7.42(1H, m) t = 3.39, [MH+] 393, 395, [MH−] 391, 393 C and Method 4 | | | |
| 27 | 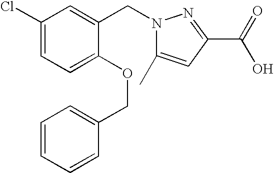 | Name NMR LCMS Method | 1-({5-chloro-2-[(phenylmethyl)oxy]phenyl}methyl)-5-meth- yl-1H-pyrazole-3-carboxylic acid ¹H NMR δ: 2.16(3H, s), 5.09(2H, s), 5.36(2H, s), 6.67(1H, s), 6.71(1H, d, J=2.5Hz), |
| #6.89(1H, d, J=8.8Hz), 7.21(1H, dd, J=8.8 and 2.5Hz), 7.36-7.43(5H, m) t = 3.35, [MH+] 357, 359, [MH−], 355, 357 C and Method 4 | | | |
| 28 | 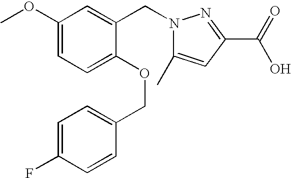 | Name NMR LCMS Method | 1-{[2-{[(4-fluorophenyl)methyl]oxy}-5-(meth- yloxy)phenyl]methyl}-5-methyl-1H-pyrazole-3-car- boxylic acid ¹H NMR δ: 2.15(3H, s), 3.68(3H, s), 5.01(2H, s), 5.36(2H, |
| #s), 6.34(1H, d, J=2.8Hz), 6.65(1H, s), 6.77(1H, dd, J=8.8 and 3.0Hz), 6.87(1H, d, J=8.8Hz), 7.06-7.11(2H, m), 7.35-7.39(2H, m) t = 3.20, [MH+] 371, [MH−] 369 D and Method 4 | | | |
| 29 | 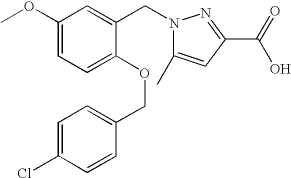 | Name NMR LCMS Method | 1-{[2-{[(4-chlorophenyl)methyl]oxy}-5-(meth- yloxy)phenyl]methyl}-5-methyl-1H-pyrazole-3-car- boxylic acid ¹H NMR δ: 2.17(3H, s), 3.68(3H, s), 5.02(2H, s), 5.37(2H, |
| #s), 6.33(1H, d, J=3.0Hz), 6.66(1H, s), 6.76(1H, dd, J=8.8 and 3.0Hz), 6.85(1H, d, J=9.0Hz), 7.32-7.38(4H, m) t = 3.36, [MH+] 387, 389, [MH−] 385, 387 D and Method 4 | | | |
| 30 | 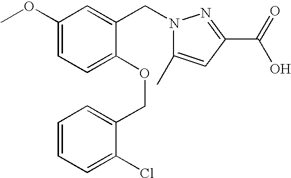 | Name NMR LCMS Method | 1-{[2-{[(2-chlorophenyl)methyl]oxy}-5-(meth- yloxy)phenyl]methyl}-5-methyl-1H-pyrazole-3-car- boxylic acid ¹H NMR δ: 2.18(3H, s), 3.68(3H, s), 5.16(2H, |
| #s), 5.42(2H, s), 6.33(1H, d, J=3.0Hz), 6.65(1H, s), 6.78(1H, dd, J=8.8 and 3.0Hz), 6.89(1H, d, J=9.0Hz), 7.29-7.31(2H, m), 7.42-7.44(1H, m), 7.47-7.49(1H, m) t = 3.36, [MH+] 387, 389, [MH−] 385, 387 D and Method 4 | | | |
| 31 | 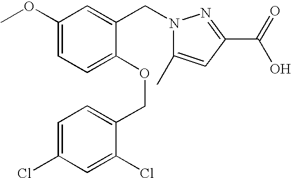 | Name NMR LCMS Method | 1-{[2-{[(2,4-dichlorophenyl)methyl]oxy}-5-(meth- yloxy)phenyl]methyl}-5-methyl-1H-pyrazole-3-car- boxylic acid ¹H NMR δ: 2.13(3H, s), 3.64(3H, s), 5.12(2H, |
| #s), 5.27(2H, s), 6.33(1H, d, J=3.0Hz), 6.46(1H, s), 6.86(1H, dd, J=9.0 and 3.3Hz), 7.04(1H, d, J=8.8Hz), 7.44(1H, dd, J=8.3 and 2.0Hz), 7.63(1H, d, J=8.3), 7.68(1H, d, J=2.0Hz) t = 3.57, [MH+] 421, 423, [MH−] 419, 421 D and Method 4 | | | |
| 32 | 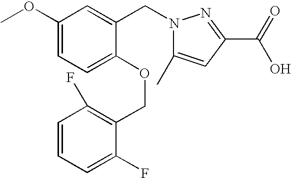 | Name NMR LCMS Method | 1-{[2-{[(2,6-difluorophenyl)methyl]oxy}-5-(meth- yloxy)phenyl]methyl}-5-methyl-1H-pyrazole-3-car- boxylic acid ¹H NMR δ: 2.12(3H, s), 3.68(3H, s), 5.13(2H, |
| #s), 5.30(2H, s), 6.34(1H, d, J=2.8Hz), 6.61(1H, s), 6.80(1H, dd, J=8.8 and 3.0Hz), 6.93-7.03(3H, m), 7.32-7.39(1H, m) t = 3.20, [MH+] 389, [MH−] 387 I2 and Method 4 | | | |
| 33 | 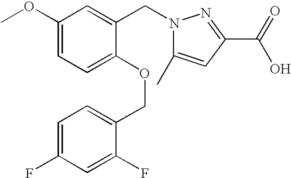 | Name NMR LCMS Method | 1-{[2-{[(2,4-difluorophenyl)methyl]oxy}-5-(meth- yloxy)phenyl]methyl}-5-methyl-1H-pyrazole-3-car- boxylic acid ¹H NMR δ: 2.10(3H, s), 3.64(3H, s), 5.08(2H, |
| #s), 5.20(2H, s), 6.33(1H, d, J=3.0Hz), 6.46(1H, s), 6.85(1H, dd, J=9.0 and 3.3Hz), 7.05-7.09(2H, m), 7.21-7.27(1H, m), 7.56-7.62(1H, m) t = 3.23, [MH+] 389, [MH−] 387 D and Method 4 | | | |
| 34 | 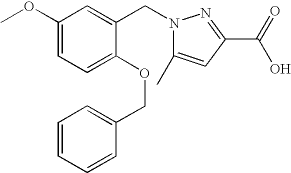 | Name NMR LCMS Method | 5-methyl-1-({5-(methyloxy)-2-[(phenyl- methyl)oxy]phenyl}methyl)-1H-pyrazole-3-car- boxylic acid ¹H NMR δ: 2.14(3H, s), 3.68(3H, s), 5.06(2H, s), 5.38(2H, s), |
| #6.34(1H, d, J=2.8Hz), 6.65(1H, s), 6.77(1H, dd, J=9.0 and 3.0Hz), 6.89(1H, d, J=9.0Hz), 7.35-7.41(5H, m) t = 3.18, [MH+] 353, [MH−] 351 D and Method 4 | | | |
| 35 | 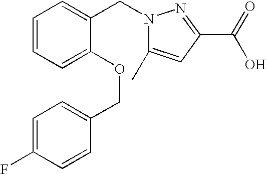 | Name NMR LCMS Method | 1-[(2-{[(4-fluorophenyl)methyl]oxy}phenyl)methyl]-5-meth- yl-1H-pyrazole-3-carboxylic acid ¹H NMR δ: 2.15(3H, s), 5.15(2H, s), 5.28(2H, s), 6.48(1H, s), 6.76(1H, d, J=6.3Hz), |
| #6.89-6.92(1H, m), 7.11(1H, d, J=8.0Hz), 7.18-7.23(1H, m), 7.25-7.29(1H, m), 7.48-7.52(1H, m) t = 3.21, [MH+] 341, [MH−] 339 E and Method 4 | | | |
| 36 | 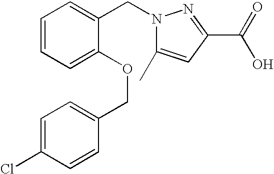 | Name NMR LCMS Method | 1-[(2-{[(4-chlorophenyl)methyl]oxy}phenyl)methyl]-5-meth- yl-1H-pyrazole-3-carboxylic acid ¹H NMR δ: 2.17(3H, s), 5.08(2H, s), 5.39(2H, s), 6.66(1H, s), 6.77(1H, d, J=7.5Hz), |
| #6.90-6.93(2H, m), 7.24-7.26(2H, m), 7.33-7.39(4H, m) t = 3.38, [MH+] 357, 359, [MH−] 355, 357 E and Method 4 | | | |
| 37 | 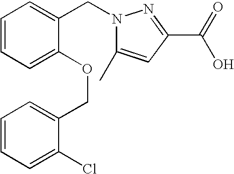 | Name NMR LCMS Method | 1-[(2-{[(2-chlorophenyl)methyl]oxy}phenyl)methyl]-5-meth- yl-1H-pyrazole-3-carboxylic acid ¹H NMR δ: 2.18(3H, s), 5.21(2H, s), 5.44(2H, s), 6.65(1H, s), 6.77(1H, d, J=7.0Hz), |
| #6.91-6.98(2H, m), 7.26-7.32(3H, m), 7.43-7.49(2H, m) t = 3.38, [MH+] 357, 359, [MH−] 355, 357 E and Method 4 | | | |
| 38 | 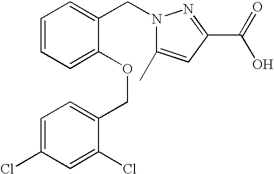 | Name NMR LCMS Method | 1-[(2-{[(2,4-di- chlorophenyl)methyl]oxy}phenyl)methyl]-5-meth- yl-1H-pyrazole-3-carboxylic acid ¹H NMR δ: 2.19(3H, s), 5.17(2H, s), 5.44(2H, s), 6.66(1H, s), |
| #6.76(1H, d, J=7.0Hz), 6.91-6.95(2H, m), 7.25-7.31(2H, m), 7.42-7.46(2H, m) t = 3.58, [MH+] 391, 393, [MH−] 389, 391 E and Method 4 | | | |
| 39 | 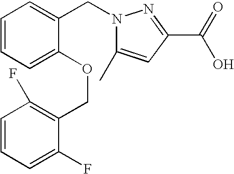 | Name NMR LCMS Method | 1-[(2-{[(2,6-di- fluorophenyl)methyl]oxy}phenyl)methyl]-5-meth- yl-1H-pyrazole-3-carboxylic acid ¹H NMR δ: 2.12(3H, s), 5.18(2H, s), 5.32(2H, |
| #s), 6.61(1H, s), 6.80(1H, d, J=6.5Hz), 6.91-6.99(3H, m), 7.08(1H, d, J=8.3Hz), 7.29-7.39(2H, m) t = 3.18, [MH+] 359, [MH−] 357 E and Method 4 | | | |
| 40 | 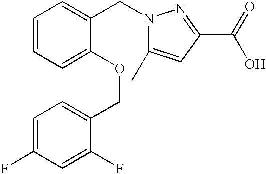 | Name NMR LCMS Method | 1-[(2-{[(2,4-di- fluorophenyl)methyl]oxy}phenyl)methyl]-5-meth- yl-1H-pyrazole-3-carboxylic acid ¹H NMR δ: 2.10(3H, s), 5.16(2H, s), 5.23(2H, |
| #s), 6.46(1H, s), 6.79(1H, d, J=7.5Hz), 6.92-6.95(1H, m), 7.09-7.18(2H, m), 7.29-7.34(2H, m), 7.61-7.67(1H, m) t = 3.25, [MH+] 359, [MH−] 357 E and Method 4 | | | |
| 41 | 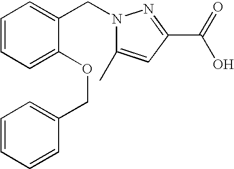 | Name NMR LCMS Method | 5-methyl-1-({2-[(phenylmethyl)oxy]phenyl}methyl)-1H-py- razole-3-carboxylic acid ¹H NMR δ: 2.15(3H, s), 5.11(2H, s), 5.41(2H, s), 6.65(1H, s), 6.78(1H, d, J=7.5Hz), |
| #6.89-6.93(1H, m), 6.97(1H, d, J=8.3Hz), 7.24-7.28(1H, m), 7.36-7.42(5H, m) t = 3.19, [MH+] 323, [MH−] 321 E and Method 4 | | | |
| 42 | 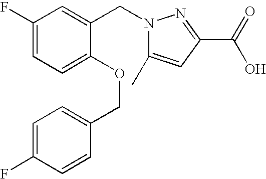 | Name NMR LCMS Method | 1-[(5-fluoro-2-{[(4-fluoro- phenyl)methyl]oxy}phenyl)methyl]-5-methyl-1H-py- razole-3-carboxylic acid ¹H NMR δ: 2.17(3H, |
| #s), 5.04(2H, s), 5.34(2H, s), 6.46(1H, dd, J=3.0Hz and 8.8Hz), 6.67(1H, s), 6.71(1H, d, J=2.5Hz), 6.87-6.90(1H, m), 6.93-6.98(1H, m), 7.08-7.12(2H, m), 7.35-7.39(2H, m) t = 3.23, [MH+] 359, [MH−] 357 F and Method 4 | | | |
| 43 | 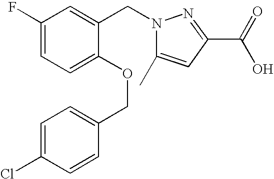 | Name NMR LCMS Method | 1-[(2-{[(4-chlorophenyl)methyl]oxy}-5-fluoro- phenyl)methyl]-5-methyl-1H-pyrazole-3-car- boxylic acid ¹H NMR δ: 2.18(3H, s), 5.05(2H, |
| #s), 5.37(2H, s), 6.42(1H, dd, J=3.0 and 8.5Hz), 6.67(1H, s), 6.83-6.87(1H, m), 6.90-6.95(1H, m), 7.32-7.39(4H, m) t = 3.38, [MH+] 375, 377, [MH−] 373, 375 F and Method 4 | | | |
| 44 | 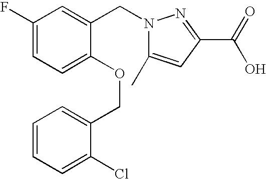 | Name NMR LCMS Method | 1-[(2-{[(2-chlorophenyl)methyl]oxy}-5-fluoro- phenyl)methyl]-5-methyl-1H-pyrazole-3-car- boxylic acid ¹H NMR δ: 2.14(3H, s), 5.21(2H, |
| #s), 5.34(2H, s), 6.49(1H, dd, J=2.8 and 8.8Hz), 6.56(1H, s), 6.98-7.08(2H, m), 7.31-7.36(2H, m), 7.44-7.47(1H, m), 7.49-7.51(1H, m) t = 3.39, [MH+] 375, 377, [MH−] 373, 375 F and Method 4 | | | |
| 45 | 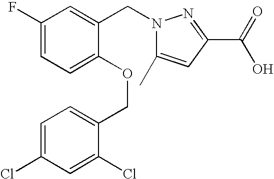 | Name NMR LCMS Method | 1-[(2-{[(2,4-dichlorophenyl)methyl]oxy}-5-fluoro- phenyl)methyl]-5-methyl-1H-pyrazole-3-car- boxylic acid ¹H NMR δ: 2.13(3H, s), |
| #5.18(2H, s), 5.28(2H, s), 6.48(1H, s), 6.59(1H, d, J=8.8Hz), 7.16(2H, d, J=5.0Hz), 7.46(1H, dd, J=8.3 and 2.0Hz), 7.64(1H, d, J=8.3Hz), 7.71(1H, d, J=2.0Hz) t = 3.59, [MH+] 409, 411, [MH−] 407, 409 F and Method 4 | | | |
| 46 | 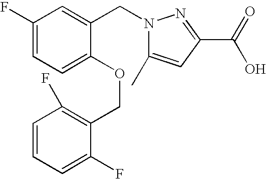 | Name NMR LCMS Method | 1-[(2-{[(2,6-difluorophenyl)methyl]oxy}-5-fluoro- phenyl)methyl]-5-methyl-1H-pyrazole-3-car- boxylic acid ¹H NMR δ: 2.13(3H, s), 5.16(2H, |
| #s), 5.30(2H, s), 6.46(1H, dd, J=8.8 and 3.0Hz), 6.64(1H, s), 6.95-7.04(4H, m), 7.34-7.41(1H, m) t = 3.21, [MH+] 377, [MH−] 375 F and Method 4 | | | |
| 47 | 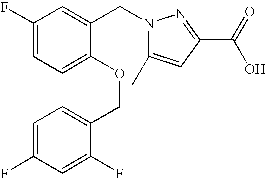 | Name NMR LCMS Method | 1-[(2-{[(2,4-difluorophenyl)methyl]oxy}-5-fluoro- phenyl)methyl]-5-methyl-1H-pyrazole-3-car- boxylic acid ¹H NMR δ: 2.18(3H, s), 5.09(2H, s), |
| #5.35(2H, s), 6.45(1H, dd, J=8.8 and 2.8Hz), 6.67(1H, s), 6.86-6.98(4H, m), 7.38-7.44(1H, m) t = 3.27, [MH+] 377, [MH−] 375 F and Method 4 | | | |
| 48 | 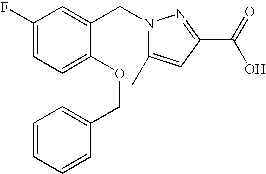 | Name NMR LCMS Method | 1-({5-fluoro-2-[(phenylmethyl)oxy]phenyl}methyl)-5-meth- yl-1H-pyrazole-3-carboxylic acid ¹H NMR δ: 2.17(3H, s), 5.08(2H, s), 5.37(2H, s), 6.44(1H, dd, J=8.8 |
| #and 2.8Hz), 6.67(1H, s), 6.87-6.96(2H, m), 7.34-7.43(5H, m) t = 3.22, [MH+] 341, [MH−] 339 F and Method 4 | | | |

The intermediate 1,1-dimethylethyl 2-[(5-chloro-2-hydroxyphenyl)ethyl]-hydrazinecarboxylate was prepared from the appropriate ketone according to Method 3.

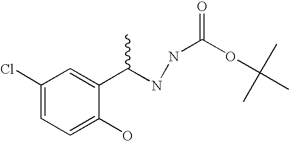

¹H NMR (CDCl₃) δ: 1.41 (3H, d, J=6.8 Hz), 1.48 (9H, s), 4.21-4.25 (1H, m), 6.23 (1H, br s), 6.77 (1H, d, J=8.6 Hz), 6.96 (1H, d, J=2.4 Hz), 7.11(1H, dd, J=8.6 J=2.3 Hz).

The intermediate 1-[1-(5-chloro-2-hydroxy-phenyl]-ethyl]-5-methyl-1H-pyrazole-3-carboxylic acid ethyl ester (G) was prepared from 1,1-dimethiethyl 2-[(5-chloro-2-hydroxyphenyl)ethyl]hydrazinecarboxylate according to Method 3.

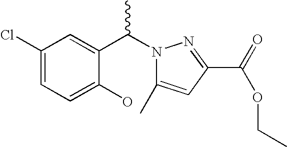

¹H NMR (CDCl₃) δ: 1.28 (3H, t, J=7.1 Hz), 1.71 (3H, d, J=6.8 Hz), 2.20 (3H, s), 4.25 (2H, dq, J=2.08 J=7.1 Hz), 5.81 (1H, q, J=6.8 Hz), 6.56 (1H, s), 6.77 (1H, d, J=2.6 Hz), 6.84 (1H, d, J=8.6 Hz), 7.14 (1H, dd, J=2.6 J=8.6 Hz), 10.15 (1H, s).

### General Method5

### 1-{1-[5-chloro-2-(2-chloro-4-fluoro-benzyloxy)-phenyl]-ethyl}-5-methyl-1H-pyrazole-3-carboxylic acid ethyl ester

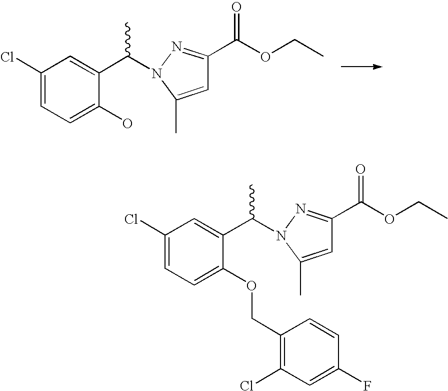

A mixture of 1-[1-(5-chloro-2-hydroxy-phenyl]-ethyl]-5-methyl-1H-pyrazole-3-carboxylic acid ethyl ester (100 mg, 0.32 mmol), K₂CO₃ (112 mg, 0.81 mmol) and 2-chloro-4-fluorobenzyl bromide (79 mg, 0.36 mmol) in acetone (3 ml) was refluxed overnight under nitrogen. After cooling the solid was filtered off and the solvent removed in vacuo. Purification was carried out on a SPE using iso-hexane containing a gradient of ethyl acetate (5-10%) to yield the title compound (120 mg, 74%).

t=3.95, [MH+] 451, 454.

The following 1H-pyrazole-3-carboxylic acid esters were prepared from G according to Method 5

| | | |
|---|---|---|
| 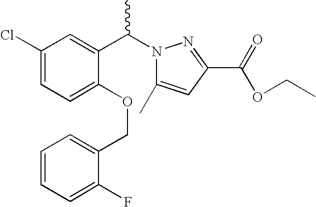 | Name LCMS Method | 1-{5-chloro-2-[(2-fluorobenzyloxy)-phenyl]-ethyl}-5-meth- yl-1H-pyrazole-3-carboxylic acid ethyl ester t = 3.78, [MH+] 417, 419 G and Method 5 |
| 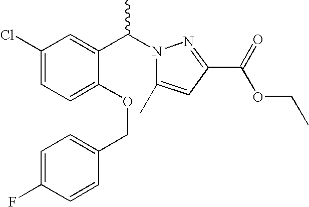 | Name LCMS Method | 1-{5-chloro-2-[(4-fluorobenzyloxy)-phenyl]-ethyl}-5-meth- yl-1H-pyrazole-3-carboxylic acid ethyl ester t = 3.77, [MH+] 417, 419 G and Method 5 |
| 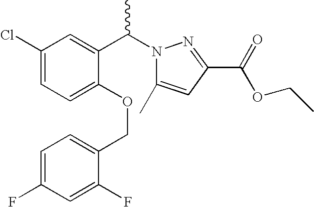 | Name LCMS Method | 1-{5-chloro-2-[(2,4-difluorobenzyloxy)-phenyl]-eth- yl}-5-methyl-1H-pyrazole-3-carboxylic acid ethyl ester t = 3.80, [MH+] 435, 437 [MH−] 433 G and Method 5 |
| 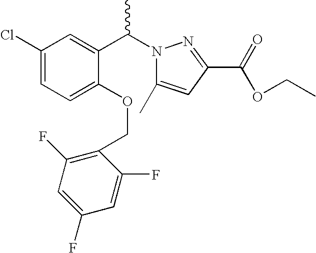 | Name LCMS Method | 1-{5-chloro-2-[(2,4,6-trifluorobenzyloxy)-phenyl]-eth- yl}-5-methyl-1H-pyrazole-3-carboxylic acid ethyl ester t = 3.58, [MH+] 453, 455 G and Method 5 |
| 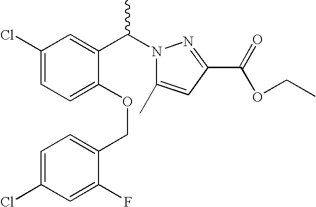 | Name LCMS Method | 1-{5-chloro-2-[(4-chloro-2-fluorobenzyloxy)-phe- nyl]-ethyl}-5-methyl-1H-pyrazole-3-carboxylic acid ethyl ester t = 3.96, [MH+] 451, 454 G and Method 5 |
| 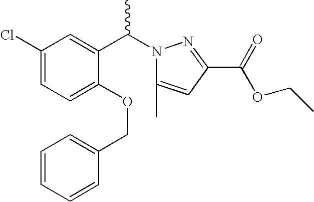 | Name LCMS Method | 1-{5-chloro-2-[(benzyloxy)-phenyl]-ethyl}-5-methyl-1H-py- razole-3-carboxylic acid ethyl ester t = 3.77, [MH+] 399, 401 G and Method 5 |

### Preparation of 1-[1-(5-chloro-2-isobutoxy-phenyl)-ethyl]-5-methyl-1H-pyrazole-3-carboxylic acid ethyl ester

A mixture of 1-[1-(5chloro-2-hydroxy-phenyl]-ethyl]-5-methyl-1H-pyrazole-3-carboxylic acid ethyl ester (100 mg, 0.32 mmol), K₂CO₃ (112 mg, 0.81 mmol) and 1-bromo-2-methylpropane (0.038 ml, 0.36 mmol) in DMF (3 ml) was heated at 80° C. under nitrogen for 2 hours. After cooling the solution was diluted with water and extracted with ethyl acetate (3×10 ml). The combined extracts were dried (MgSO₄) and evaporated. Purification was carried out on a SPE (20% ethyl acetate:isohexane) to yield the title compound.

t=3.88, [MH+] 365, 367.

### General Method6

### Example 49

### 1-{1-[5-chloro-2-(2-chloro-4-fluoro-benzyloxy)-phenyl]-ethyl}-5-methyl-1H-pyrazole-3-carboxylic acid

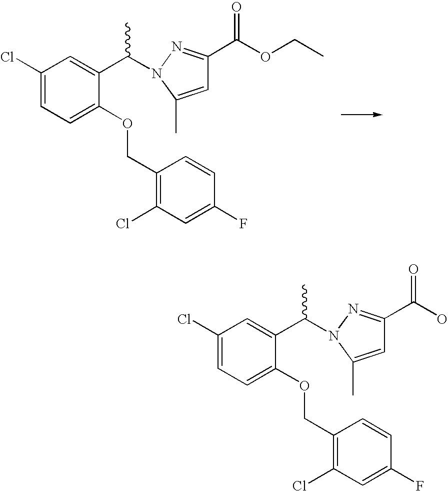

To a solution of 1-{1-[5-chloro-2-(2-chloro-4-fluoro-benzyloxy)-phenyl]-ethyl}-5-methyl-1H-pyrazole-3-carboxylic acid ethyl ester (120 mg, 0.26 mmol) in 3 ml of ethanol and 1 ml of water, NaOH (42 mg, 1.06 mmol) was added. The mixture was stirred at 60° C. for 2 hours. the solution was diluted with water, acidified with acetic acid and extracted with ethyl acetate. The organic solution was dried over MgSO₄ and evaporated to give the title compound (112 mg, 99%).

¹H NMR (DMSO) δ: 1.67 (3H, bs), 1.94 (3H, s), 5.21 (2H, s), 5.66 (1H, q, J=6.8 Hz), 6.12 (1H, s), 6.82 (1H, d, J=2.6 Hz), 7.18 (1H, d, J=8.8 Hz), 7.19-7.31 (2H, m), 7.56 (1H, dd, J=2.6 J=8.8 Hz), 7.64 (1H, m).

t=3.76, [MH−] 421, 424.

The following Examples were prepared from the appropriate ester intermediate according to Method 6

| | | | |
|---|---|---|---|
| 50 | 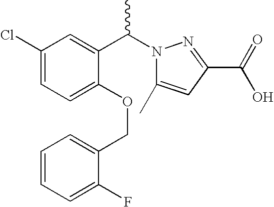 | Name NMR LCMS Method | 1-{5-chloro-2-[(2-fluorobenzyloxy)-phenyl]-ethyl}-5-meth- yl-1H-pyrazole-3-carboxylic acid ¹H NMR δ: (DMSO) 1.68(3H, d, J=6.8Hz), |
| #2.01(3H, s), 5.17-5.24(2H, m), 5.77(1H, q, J=6.8Hz), 6.43(1H, s), 6.93(1H, d, J=2.6Hz), 7.2-7.5(6H, m), 12.6(1H, bs). t = 3.59, [MH+] 389 [MH−] 387, 389 Method 6 | | | |
| 51 | 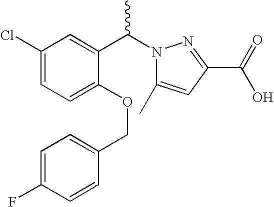 | Name NMR LCMS Method | 1-{5-chloro-2-[(4-fluorobenzyloxy)-phenyl]-ethyl}-5-meth- yl-1H-pyrazole-3-carboxylic acid ¹H NMR δ: (DMSO) 1.68(3H, d, |
| #J=6.8Hz), 2.0(3H, s), 5.17(2H, s), 5.73(1H, q, J=6.8Hz), 6.26(1H, s), 6.88(1H, d, J=2.6Hz), 7.13(1H, d, J=8.8Hz), 7.12-7.32(3H, m), 7.46-7.5(2H, m). t = 3.56, [MH+] 3.89 [MH−] 387, 389 Method 6 | | | |
| 52 | 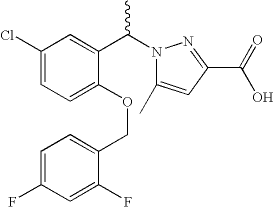 | Name NMR LCMS Method | 1-{5-chloro-2-[(2,4-difluorobenzyloxy)-phenyl]-eth- yl}-5-methyl-1H-pyrazole-3-carboxylic acid ¹H NMR δ: (DMSO) 1.66(3H, d, |
| #J=6.8Hz), 2.0(3H, s), 5.2(2H, s), 5.63(1H, q, J=6.8Hz), 6.14(1H, s), 6.82(1H, d, J=2.6Hz), 7.12-7.21(2H, m), 7.28-7.37(2H, m), 7.60(1H, q, J=6.8Hz), t = 3.61, [MH+] 407, 409 [MH−] 405, 407 Method 6 | | | |
| 53 | 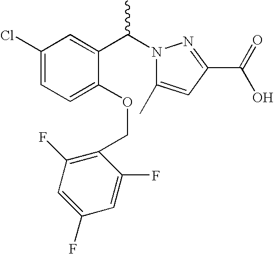 | Name NMR LCMS Method | 1-{5-chloro-2-[(2,4,6-trifluorobenzyloxy)-phenyl]-eth- yl}-5-methyl-1H-pyrazole-3-carboxylic acid ¹H NMR δ: (DMSO) 1.63(3H, d, J=6.8Hz), |
| #1.96(3H, s), 5.14(2H, q, J=11Hz), 5.65(1H, q, J=6.8Hz), 6.37(1H, s), 6.96(1H, s), 7.25-7.39(4H, m). t = 3.58, [MH+] 425, 427 [MH−] 423, 425 Method 6 | | | |
| 54 | 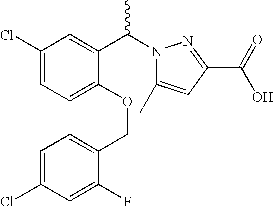 | Name NMR LCMS Method | 1-{5-chloro-2-[(4-chloro-2-fluorobenzyloxy)-phe- nyl]-ethyl}-5-methyl-1H-pyrazole-3-carboxylic acid ¹H NMR δ: |
| #(DMSO) 1.68(3H, d, J=6.8Hz), 1.97(3H, s), 5.21(2H, s), 5.67(1H, q, J=6.8Hz), 6.18(1H, s), 6.82(1H, d, J=2.6Hz), 7.17(1H, d, J=8.8Hz), 7.24-7.38(2H, m), 7.50-7.62(2H, m). t = 3.83, [MH−] 421 Method 6 | | | |
| 55 | 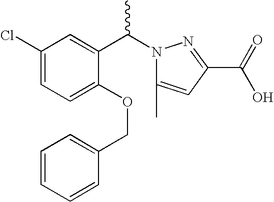 | Name NMR LCMS Method | 1-{5-chloro-2-[(benzyloxy)-phenyl]-ethyl}-5-methyl-1H-py- razole-3-carboxylic acid ¹H NMR δ: (DMSO) 1.68(3H, bs), 2.1(3H, s), 5.21(2H, |
| #s), 5.75(1H, q, J=6.8Hz), 6.2(1H, s), 6.82(1H, d, J=2.6Hz), 7.15(1H, d, J=8.8Hz), 7.21-7.5(6H, m). t = 3.58, [MH+] 371 Method 6 | | | |
| 56 | 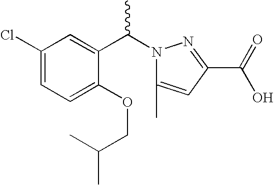 | Name NMR LCMS Method | 1-[1-(5-chloro-2-isobutoxy-phenyl)-ethyl]-5-methyl-1H-py- razole-3-carboxylic acid ¹H NMR δ: (DMSO) 0.98(6H, t, J=7.08Hz), |
| #1.71(3H, d, J=6.88Hz), 2.02-2.06(1H, m), 2.18(3H, s), 3.80(2H, d, J=6.36Hz), 5.83-5.87(1H, m), 6.52(1H, s), 6.85(1H, d, J=2.6Hz), 7.04(1H, d, J=8.8Hz), 7.3(1H, dd, J=8.7, J=2.6Hz), 12.6(1H, bs). t = 3.65, [MH+] 337 Method 6 | | | |

### Preparation of 1-(5-chloro-2-hydroxy-benzyl)-5-methyl-1H-pyrrole-3-carboxylic acid ethyl ester (H)

### Preparation of 2-benzyloxy-5-chloro-benzamide

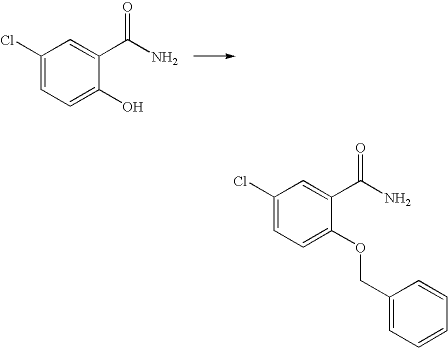

A mixture of 5chloro-2-hydroxy-benzamide (8 g, 0.046 mol), K₂CO₃ (7.72 g, 0.056 mol) and benzyl bromide (6.1 ml, 0.051 mol) in acetone (50 ml) was refluxed overnight, under nitrogen. After cooling, the solid was filtered off and the filtrate was cooled (in a fridge) to effect crystallisation. The resultant solid was collected to give 9.9 g (81%) of a colourless solid.

t=2.90, [MH+] 262, 264

### Preparation of 2-benzyloxy-5chloro-benzylamine

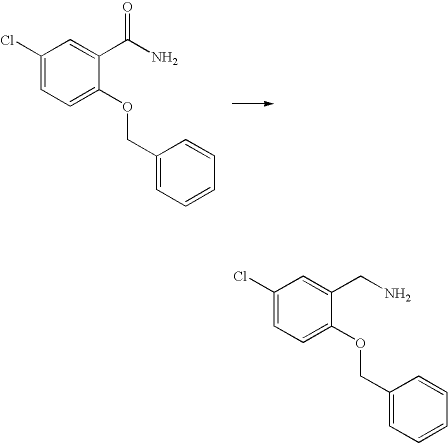

2-benzyloxy-5chloro-benzamide (7.9 g, 0.030 mol) in 20 ml of tetrahydrofuran was slowly added, under nitrogen, to a 1M solution of LiAlH₄ (45 ml) in tetrahydrofuran at 0° C. The reaction mixture was then heated at 70° C. for 1 hour. After cooling the reaction mixture was poured onto water and extracted with ethyl acetate (3×40 ml). The combined extracts were dried (MgSO₄)and evaporated to give the title compound as a yellow oil (7 g, 94%).

¹H NMR δ: 1.66 (2H, bs), 3.84 (2H, s), 5.07 (2H, s), 6.83 (1H, d, J=8.6 Hz), 7.15 (1H, dd, J=8.6 and 2.6 Hz), 7.24-7.42 (6H, m).

### Preparation of 4,4-dimethoxy-pentanoic acid methyl ester

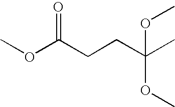

Ethyl levulinate (20 g, 0.138 mol), trimethyl orthoformate (15.3 g, 0.144 mol) and a catalytic amount of p-toluene sulfonic acid monohydrate in 6 ml of methanol were refluxed over the weekend. After cooling the mixture was vacum down and the residue used with no further purifications.

¹H NMR δ: 1.25 (3H, bs), 1.94-1.98 (2H, m), 2.32-2.37 (2H,m), 3.17 (6H, s), 3.68 (3H, s).

### Preparation of 2-formyl-4-oxo-pentanoic acid ethyl ester

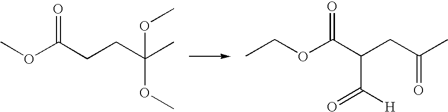

A mixture of 4,4-dimethoxy-pentanoic acid methyl ester (25 g, 0.13 mol) and ethyl formate (21 ml, 0.26 mol), was added to a solution of NaH (5.78 g, 0.144 mol) in THF (50 ml) at ˜10° C. The reaction mixture was stirred for 3 h, then let stand overnight. Water (100 ml) and ether(60 ml) were added and the mixture stirred for 5 minutes. The organic phase was then separated and washed with water. The combined water layers were acidified to pH2 and extracted with ethyl acetate (3×50 ml). The combined extracts were dried (MgSO₄) and evaporated. The residue was then distilled, the fraction with b.p. 110-120° C. was the desired compound.

¹H NMR δ: 1.27-1.32 (3H, m), 2.23 (3H, s), 2.63 (1H, t, J=6.7 Hz), 2.76 (1H, t, J=6.7 Hz), 3.78-3.81 (1H, m), 4.19-4.28 (2H, m), 9.93 (1H, s).

### Preparation of 1-(2-benzyloxy-5-chloro-benzyl)-5-methyl-1H-pyrrole-3 carboxylic add ethyl ester

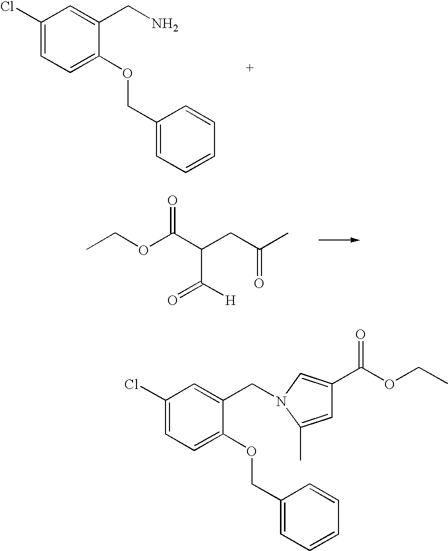

To a mixture of 2-formyl-4-oxo-pentanoic acid ethyl ester (2.5 g, 0.016 mol) and 2-benzyloxy-5-chloro-benzylamine (4.7 g, 0.019 mol), CH₃COOH (˜3 ml) was added. The reaction mixture was stirred for 2 hours then was poured onto water and extracted with ethyl acetate (3×40 ml). The combined extracts were dried (MgSO₄) and evaporated. The residue was purified on a Biotage (15% ethyl acetate:iso-hexane) to give the title compound as a yellow solid (2.8 g, 45%).

¹H NMR δ: 1.32 (3H, t, J=7.1 Hz), 2.08 (3H, s), 4.25 (2H, q, J=7.1 Hz), 4.98 (2H, s), 5.07 (2H, s), 6.35 (1H, s), 6.61 (1H, s), 6.87 (1H, d, J=8.7 Hz), 7.18-7.21 (2H, m), 7.33-7.41 (5H, m).

### Preparation of 1-(5-chloro-2-hydroxy-benzyl)-5-methyl-1H-pyrrole-3-carboxylic acid

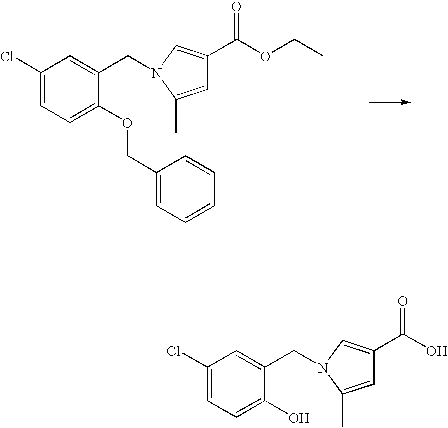

A mixture of sodium methanethiolate (1.16 g,16.5 mmol) and 1-(2-benzyloxy-5-chloro-benzyl)-5-methyl-1H-pyrrole-3 carboxylic acid ethyl ester (1.27 g, 3.3 mmol) in DMF(14 ml) was stirred at 100° C. for 3 hours. After cooling the mixture was diluted with water and acidified with 1M HCl and then extracted with ethyl acetate. The organic phase was dried (MgSO₄), evaporated to dryness to give the title compound as a yellow oil.

t=2.76, [MH+] 266 [MH−] 264.

### Preparation of 1-(5-chloro-2-hydroxy-benzyl)-5-methyl-1H-pyrrole-3-carboxylic acid ethyl ester

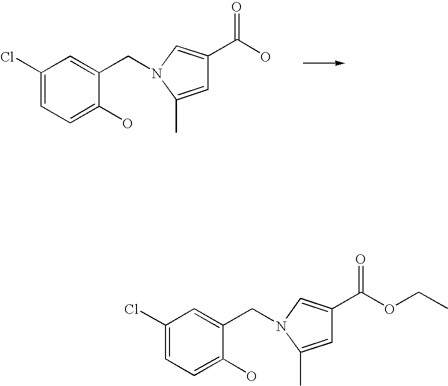

A mixture of 1-(5-chloro-2-hydroxy-benzyl)-5-methyl-1H-pyrrole-3-carboxylic acid (3.3 mmol) and H₂SO₄ (1.5 ml) in ethanol (15 ml) was refluxed overnight.

After cooling the mixture was diluted with water basified with NH₃ and then extracted with ethyl acetate (3×20 ml). The combined organic layers were dried (MgSO₄), and the solvent removed in vacuo. Purification was carried out on a SPE using 30% ethyl acetate in iso-hexane to yield the title compound as a yellow solid (0.73 g,75%).

t=3.28, [MH+] 294,296 [MH−] 292.

The following intermediates were prepared from 1-(5-chloro-2-hydroxy-benzyl)-5-methyl-1H-pyrrole-3-carboxylic acid ethyl ester (intermediate H) according to Method 5.

| | | |
|---|---|---|
| 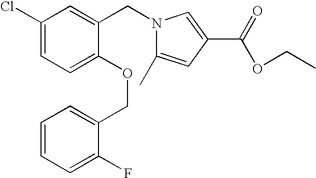 | Name LCMS Method | 1-[5-chloro-2-(2-fluoro-benzyloxy)-benzyl]-5-meth- yl-1H-pyrrole-3-carboxylic acid ethyl ester t = 3.92, [MH+] 402, 404 H and Method 5 |
| 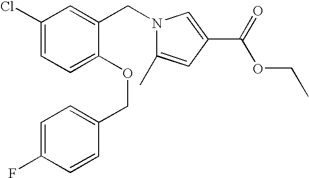 | Name LCMS Method | 1-[5-chloro-2-(4-fluoro-benzyloxy)-benzyl]-5-meth- yl-1H-pyrrole-3-carboxylic acid ethyl ester t = 3.91, [MH+] 402, 404 H and Method 5 |
| 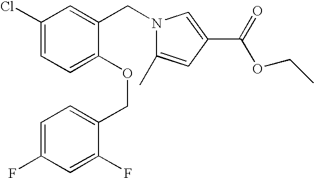 | Name LCMS Method | 1-[5-chloro-2-(2,4-difluoro-benzyloxy)-benzyl]-5-meth- yl-1H-pyrrole-3-carboxylic acid ethyl ester t = 3.93, [MH+] 420, 422 H and Method 5 |
| 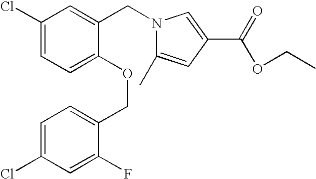 | Name LCMS Method | 1-[5-chloro-2-(4-chloro-2-fluoro-benzyloxy)-ben- zyl]-5-methyl-1H-pyrrole-3-carboxylic acid ethyl ester t = 4.09, [MH+] 436, 439 H and Method 5 |
| 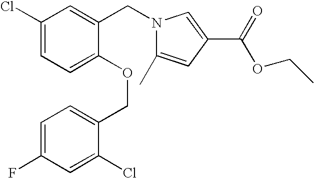 | Name LCMS Method | 1-[5-chloro-2-(2-chloro-4-fluoro-benzyloxy)-ben- zyl]-5-methyl-1H-pyrrole-3-carboxylic acid ethyl ester t = 4.08, [MH+] 436, 439 H and Method 5 |

The following Examples were prepared from the appropriate ester intermediate according to Method 6.

| | | | |
|---|---|---|---|
| 57 | 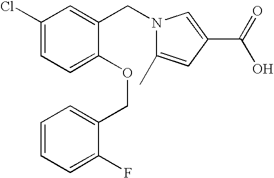 | Name NMR LCMS Method | 1-[5-chloro-2-(2-fluoro-benzyloxy)-benzyl]-5-meth- yl-1H-pyrrole-3-carboxylic acid ¹H NMR (DMSO) δ: 1.99(3H, s), 5.03(2H, s), 5.23(2H, s), 6.15(1H, s), 6.62(1H, s), 7.21-7.29(5H, m), 7.36(1H, d, J=8Hz), 7.41-7.45(1H, m), |
| #7.53(1H, t, J=7.4Hz), 11.6(1H, s). t = 3.50, [MH+] 374, 376 [MH−] 372, 374 Method 6 | | | |
| 58 | 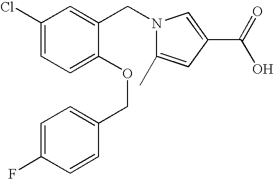 | Name NMR LCMS Method | 1-[5-chloro-2-(4-fluoro-benzyloxy)-benzyl]-5-meth- yl-1H-pyrrole-3-carboxylic acid ¹H NMR (DMSO) δ: 2.03(3H, s), 5.07(2H, s), 5.17(2H, s), 6.17(1H, s), 6.64(1H, s), 7.14-7.23(3H, m), 7.29(1H, s), 7.33(1H, bd), |
| #7.47-7.5(2H, m), 11.59(1H, s). t = 3.48, [MH+] 374, 376 [MH−] 372, 374 Method 6 | | | |
| 59 | 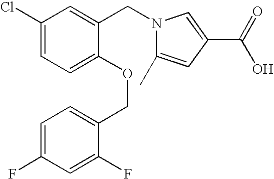 | Name NMR LCMS Method | 1-[5-chloro-2-(2,4-difluoro-benzyloxy)-benzyl]-5-meth- yl-1H-pyrrole-3-carboxylic acid ¹H NMR (DMSO) δ: 1.99(3H, s), 5.02(2H, s), 5.19(2H, s), 6.15(1H, s), 6.64(1H, s), 7.08-7.14(1H, m), 7.21-7.38(4H, m), 7.58-7.65(1H, |
| #m), 11.6(1H, s). t = 3.48, [MH+] 392, 394 [MH−] 390, 392 Method 6 | | | |
| 60 | 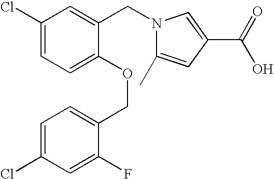 | Name NMR LCMS Method | 1-[5-chloro-2-(4-chloro-2-fluoro-benzyloxy)-ben- zyl]-5-methyl-1H-pyrrole-3-carboxylic acid ¹H NMR (DMSO) δ: 2.0(3H, s), 5.03(2H, s), 5.21(2H, s), 6.16(1H, s), 6.63(1H, s), 7.21(1H, d, J=8.8Hz), 7.28(1H, s), |
| #7.31-7.37(2H, m), 7.5-7.58(2H, m), 11.6(1H, s). t = 3.70, [MH+] 408, 411 [MH−] 406, 410 Method 6 | | | |
| 61 | 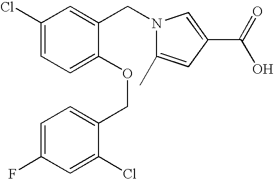 | Name NMR LCMS Method | 1-[5-chloro-2-(2-chloro-4-fluoro-benzyloxy)-ben- zyl]-5-methyl-1H-pyrrole-3-carboxylic acid ¹H NMR (DMSO) δ: 1.99(3H, s), 5.06(2H, s), 5.21(2H, s), 6.16(1H, s), 6.63(1H, s), 7.19-7.28(3H, m), 7.37(1H, bd), 7.55(1H, |
| #bd), 7.6-7.68(1H, m), 11.6(1H, s). t = 3.70, [MH+] 408, 411 [MH−] 406, 409 Method 6 | | | |

It is to be understood that the present invention covers all combinations of particular and preferred subgroups described herein above.

Assays for Determining Biological Activity

The compounds of formula (I) can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

Biological Activity at EP₁ and EP₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-3, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing fluo-3 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ (C³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20 mins and the pellet containing the membrane fraction is washed three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques (GraphPad Prism 3) to determine the concentration of compound producing 50% inhibition of specific binding (IC₅₀).

Biological Activity at TP Receptor

To determine if a compound has agonist or antagonist activity at the TP receptor a functional calcium mobilisation assay may be performed. Briefly, the antagonist properties of compounds are assessed by their ability to inhibit the mobilisation of intracellular calcium ([Ca²⁺]ᵢ) in response to activation of TP receptors by the stable TXA₂ mimetic U46619. Increasing concentrations of antagonist reduce the amount of calcium that a given concentration of U46619 can mobilise. The net effect is to displace the U46619 concentration-effect curve. The amount of calcium produced is assessed using a calcium-sensitive fluorescent dye such as Fluo-3, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software. The agonist activity of the compounds are determined by their ability to cause an increase in intracellular mobilisation in the absence of U46619.

The human TP calcium mobilisation assay utilises Chinese hamster ovary-K1 (CHO-K1) cells into which a stable vector containing TP cDNA has previously been transfected. Cells are cultured in suitable flasks containing culture medium such as DMEM:F-12 supplemented with 10% v/v foetal calf serum, 2 mM L-glutamine, 0.25 mg/ml geneticin and 10 μg/ml puromycin.

For assay, cells are harvested using a proprietary reagent that dislodges cells such as Versene. Cells are re-suspended in a suitable quantity of fresh culture media for introduction into a 96-well plate. Following incubation for 24 hours at 37° C. the culture media is replaced with a medium containing fluo-3 and the detergent pluronic acid, and a further incubation takes place. Concentrations of compounds are then added to the plate in order to construct concentration-effect curves. This may be performed on the FLIPR in order to assess the agonist properties of the compounds. Concentrations of U46619 are then added to the plate in order to assess the antagonist properties of the compounds.

The data so generated may be analysed by means of a computerised curve-fitting routine. The concentration of compound that elicits a half-maximal inhibition of the calcium mobilisation induced by U46619 (pIC50) may then be estimated, and the percentage activation caused by the compounds directly can be used to determine if there is any agonism present.

By application of these techniques, compounds of the Examples had an antagonist binding pIC₅₀ value of 6.2-9.9 at EP₁ receptors and a pIC₅₀ value of <5.7 at EP₃ receptors. The compounds of the examples had a functional pKi of 6.2-10.5 and/or a functional pIC₅₀ of 5.3-8.9.

No toxicological effects are indicated/expected when a compound (of the invention) is administered in the above mentioned dosage range.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 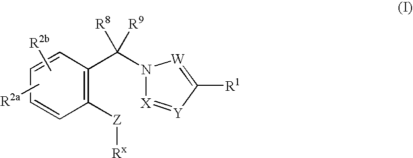
wherein:
W represents N or CR¹⁰ wherein R¹⁰ represents hydrogen, halogen, optionally substituted alkyl, optionally substituted aryl, or optionally substituted heterocyclyl;
X represents N or CR¹¹ wherein R¹¹ represents hydrogen, halogen, optionally substituted alkyl, optionally substituted aryl, or optionally substituted heterocyclyl;
Y represents N or CR¹² wherein R¹² represents hydrogen, halogen, CH₃ or CF₃;
Z represents O, S, SO or SO₂;
R¹ represents CO₂R⁴, CONR⁵R⁶, CH₂CO₂H, optionally substituted SO₂alkyl, SO₂NR⁵R⁶, NR⁵CONR⁵R^{6,} 2H-tetrazol-5-yl-methyl or optionally substituted heterocyclyl;
R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2: or R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ are independently selected from hydrogen, fluorine or alkyl, or R⁸ and R⁹ together with the carbon to which they are attached form a cycloalkyl ring, optionally containing up to one heteroatom selected from O, S, NH or N-alkyl;
wherein Q^{a} and Q^{b} are each independently selected from hydrogen, CH₃ and fluorine;
or a derivative thereof.

2. A compound according to claim 1 wherein the five membered ring comprising W, X and Y is pyrrole or pyrazole.

3. A compound according to claim 1 wherein R¹ is CO₂H.

4. (canceled)

5. A pharmaceutical composition comprising a compound according to of claims 1 or a pharmaceutically acceptable derivative thereof together with a pharmaceutical carrier and/or excipient.

6.-7. (canceled)

8. A method of treating a human or animal subject suffering from a condition which is mediated by the action of PGE₂ at EP₁ receptors which comprises administering to said subject an effective amount of a compound according to claims 1 or a pharmaceutically acceptable derivative thereof.

9. A method of treating a human or animal subject suffering from inflammatory pain, neuropathic pain or visceral pain which method comprises administering to said subject an effective amount of a compound according to claims 1 or a pharmaceutically acceptable derivative thereof.

10.-11. (canceled)

12. The method of claim 8, wherein the subject is a human.

13. The method of claim 9, wherein the subject is a human.

14. A method of mediating EP₁ receptors, comprising the step of administering an effective amount of a compound according to claim 1 or a pharmaceutically acceptable derivative thereof.
